(19) United States
(12) Patent Application Publication
(10) Pub. No.: US 2005/0137251 A1
(43) Pub. Date: Jun. 23, 2005

(54) Dexanabinol and dexanabinol analogs regulate inflammation related genes

(76) Inventors: Aaron Garzon, Rehovot (IL); Ayelet Avraham, Gedera (IL); George Fink, Tel Aviv (IL)

Correspondence Address: WINSTON & STRAWN LLP, 1700 K STREET, N.W., WASHINGTON, DC, 20006, US

(21) Appl. No.: 10/942,504

(22) Filed: Sep. 16, 2004

Related U.S. Application Data
(63) Continuation of application No. PCT/IL03/00223, filed on Mar. 16, 2003.

(30) Foreign Application Priority Data
Mar. 18, 2002 (IL) IL 148736

(51) Int. Cl. A61K 31/353

(52) U.S. Cl. 514/454

(57) ABSTRACT

The present invention relates to methods of treatment utilizing pharmaceutical compositions that include as active ingredients non-psychotropic cannabinoid derivatives that modulate the expression of genes involved in inflammatory and immune processes. Regulating the transcription of pro and anti-inflammatory mediators has useful therapeutic application for prevention and treatment of acute and chronic inflammation, autoimmune diseases and related disorders, pain, infections, liver diseases, cardiovascular disorders, gastrointestinal disorders, disorders of the central and peripheral nervous system including neurodegenerative diseases, respiratory diseases, renal diseases, post-operative complications, tissue rejection and certain types of cancer.

### CROSS-REFERENCE TO RELATED APPLICATIONS

This application is a continuation of International application PCT/IL2003/000223 filed Mar. 16, 2003, the entire content of which is expressly incorporated herein by reference thereto.

### FIELD OF THE INVENTION

The present invention relates to pharmaceutical compositions comprising as an active ingredient non-psychotropic cannabinoid derivatives that modulate the expression of genes involved in inflammatory and immune processes. Regulating the transcription of pro and anti-inflammatory mediators has useful therapeutic application for prevention and treatment of acute and chronic inflammation, autoimmune diseases and related disorders, pain, infections, liver diseases, cardiovascular disorders, gastrointestinal disorders, disorders of the central and peripheral nervous system including neurodegenerative diseases, respiratory diseases, renal diseases, post-operative complications, tissue rejection and certain types of cancer.

### BACKGROUND OF THE INVENTION

### Cannabinoids

Cannabis sativa preparations have long been known as therapeutic agents to treat various diseases. The identification of tetrahydrocannabinol (THC) as the active principle of marijuana prompted medicinal chemists to develop numerous cannabinoid analogs. These novel compounds were designed to exhibit the therapeutically beneficial properties of THC without the clinically undesirable psychotropic effects. Potential therapeutic applications have classically included known attributes of marijuana itself such as anti-emesis, analgesia, anti-glaucoma and appetite stimulation. More recently recognized roles for non-psychotropic cannabinoids are as neuroprotective and anti-inflammatory agents. The diverse cannabinoid effects are generally attributed to the activation or inhibition of various types of receptors. Nevertheless, the mechanisms underlying some therapeutic effects of cannabinoid derivatives remain unclear.

Several synthetic cannabinoid derivatives have been prepared since the discovery of the natural ligand with an emphasis toward therapeutic non-psychoactive agents. An extended family of such compounds were disclosed for example in U.S. Pat. Nos. 4,179,517, 4,876,276, 5,538,993, 5,635,530, 6,096,740, and in international patent application WO 01/98289. A pivotal member of this family of compounds is 1,1-dimethylheptyl-(3S,4S)-7-hydroxy-Δ⁶-tetrahydrocannabinol, disclosed in U.S. Pat. No. 4,876,276 and denoted therein as HU-211. HU-211 was subsequently assigned the trivial chemical name dexanabinol. The neuroprotective action of dexanabinol, and later on its derivatives, was attributed in part to their ability to block the NMDA receptor. Moreover, the compounds seemed to share anti-oxidative and anti-inflammatory properties unrelated to their capacity to block the NMDA receptor. This anti-inflammatory activity was associated with the ability of those compounds to reduce the secretion of PGE₂ produced by the enzyme cyclooxygenase-2 (COX-2). COX-2 is one of the cyclooxygenase isoforms involved in the metabolism of arachidonic acid (AA) toward prostaglandins (PG) and other eicosanoids, a family of compounds known to exhibit inflammatory properties and known to be involved in inflammation. Most conventional NSAIDS (non-steroidal anti-inflammatory drugs) inhibit COX activity by modifying the enzyme active site thereby preventing the transformation of the AA substrate to PGE₂ (Hinz B. et al., J. Pharm. Exp. Ther. 300: 367- 375, 2002).

Moreover, dexanabinol and later on its analogs were found able to block the production or action of TNF-α, as disclosed in International Patent applications WO 97/11668 and WO 01/98289. It was postulated that the inhibition of the cytokine occurs at a post-transcriptional stage, since in a model of head injury dexanabinol did not affect the levels of TNF-α mRNA (Shohami E. et al., J. Neuroimmuno. 72: 169-77, 1997). Human TNF-α is first translated into a 27 kd transmembrane precursor protein, which is cleaved into the secreted 17 kd form by TNF-α converting enzyme (TACE). Based on RT-PCR experiments, Shoshany et al. reported that dexanabinol has no significant effect on TNF-α mRNA whereas it significantly reduced the levels of TACE mRNA, supporting the assumption that the drug acts at the level of secretion inhibition.

### Inflammation

Inflammation is one of the most important processes involved in the defense of an organism; however, when it occurs in some organs, such as brain, in response to an insult, or if it is inappropriate, such as in autoimmune diseases, inflammation can be harmful and therefore requires pharmacological treatment. The inflammatory response involves many effector mechanisms that produce a multiplicity of vascular and cellular reactions. Vasodilatation, increased microvascular permeability, chemotaxis, cellular activation, pain and finally repair are mediated by the local production and release of several specific mediators. Cytokines, chemokines, arachidonic acid derivatives (prostaglandins, thromboxanes and leukotrienes), oxygen and possibly nitrogen radicals, play a regulatory role in this complex and highly balanced process. Following is a selection of genes whose protein product was shown to be involved in a variety of pathways leading to inflammation: COX-2, IL-2, MCP-1, IL-1β, iNOS, TNF-α, IL-6 and IL-10 and SOCS.

The involvement of these inflammatory mediators in various pathological conditions was extensively reviewed (Frangogianis N. G. et al., Cardio. Res. 53: 31-47, 2002; Gerard C. et al., Nature Immuno. 2: 108-15, 2001; Nathan C., Nature 420: 846-852, 2002; Quan N. et al., Histol. Histopathol. 17: 273-88, 2002). Blocking the effect of these inflammatory mediators by diverse strategies includes, in addition to the conventional small molecule drug approach, the preparation of antibodies, peptidic mimetics or decoys, oligonucleotides, either antisense or triple-helix forming, directed either against the ligand or against its receptor(s). Selective inhibition of COX-2 alone is disclosed for example in U.S. Pat. Nos. 5,783,597, 5,840,746 and 6,025,253 which provide small molecules for alleviating, preventing or treating inflammation, neurodegenerative diseases and angiogenic disorders. U.S. Pat. No. 6,344,323 discloses COX-2 antisense oligonucleotides and methods of use thereof and U.S. Pat. No. 5,776,502 discloses methods of transcriptional modulation of various genes including inflammatory mediators using molecules that bind to DNA, RNA or protein. The use of antibodies or peptides is exemplified in U.S. Pat. No. 6,277,969 which discloses antibodies to human TNF-α and peptides thereof for diagnosis and treatment of pathologies involving this cytokine.

Despite the progress in this field, there remains an unmet medical need for effective therapies for inflammatory diseases. The pro-inflammatory and anti-inflammatory mediators have pleiotropic effects, including the property of regulating one another. Therefore it is not surprising that the previously described cytokines, cytokine regulators, chemokines and “pro-inflammatory” enzymes are involved in numerous diseases where they can be either deleterious or beneficial. When appropriately regulated and balanced, these agents protect the host by activating defense mechanisms and therefore their complete inhibition is not desirable. However, if the inflammation is inappropriate and the expression of those mediators is highly dysregulated, then tissue damage may result. Compounds that would selectively and simultaneously down regulate pro-inflammatory mediators and up-regulate anti-inflammatory ones without totally blocking their physiological beneficial effects would have a clear therapeutic benefit for a wide range of disease states.

### SUMMARY OF THE INVENTION

The present invention relates to pharmaceutical compositions comprising as an active ingredient non-psychotropic cannabinoids and their derivatives that are now disclosed unexpectedly to act as direct or indirect regulators of genes involved in inflammatory mechanisms. The present invention encompasses any synthetic or natural cannabinoid which is essentially devoid of appreciable psychomimetic activity. Currently preferred are synthetic non-psychotropic derivatives of dexanabinol, also known as HU-211.

Surprisingly, it is now disclosed that the PGE₂ inhibitory activity displayed by the preferred compounds does not occur at the level of the COX-2 enzymatic activity, but rather at the level of gene regulation. Therefore, some novel non-psychotropic cannabinoids are useful for the treatment of acute and chronic inflammation, autoimmune diseases and related disorders, pain, infections, liver diseases, cardiovascular disorders, gastrointestinal disorders, disorders of the central and peripheral nervous system including neurodegenerative diseases, respiratory diseases, renal diseases, post-operative complications, tissue rejection and certain types of cancer through means not previously envisioned. The fact that compounds of the invention act at the level of gene transcription by down-regulating pro-inflammatory mediators or by up-regulating anti-inflammatory ones or by having both activities simultaneously serve as a basis for treating a wide range of conditions with said compounds.

The present invention encompasses pharmaceutical compositions for decreasing the transcription of at least one of the pro-inflammatory mediators COX-2, IL-1β, IL-2, iNOS, TNF-α and MCP-1, comprising as an active ingredient a compound of general formula (I): 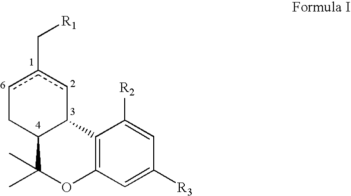
having the (3S,4S) configuration and being essentially free of the (3R,4R) enantiomer, wherein the dashed line indicates an optional C1-C2 or C6-C1 double bond, and wherein:
R′ is selected from the group consisting of
a) R′ where R′ is selected from the group consisting of
   A) a linear or branched, saturated or unsaturated, carbon side chain comprising 1-8 carbon atoms optionally interrupted by 1-3 heteroatoms, and
   B) a saturated or unsaturated cyclic moiety, an aromatic moiety or a heterocyclic moiety; the cyclic moiety having from 5-20 atoms comprising one or two-ringed structures, wherein each ring comprises 3-8 carbons, optionally interrupted by 1-4 heteroatoms, and optionally further substituted with one or more groups selected from
      i) a linear, branched or cyclic, saturated or unsaturated C₁-C₆ alkyl,
      ii) a linear, branched or cyclic, saturated or unsaturated C₁-C₆ alkoxy,
      iii) a linear, branched or cyclic, saturated or unsaturated C₁-C₆ alkylthio,
      iv) a halogen,
      v) carboxyl,
      vi) —CO₂—C₁-C₄ alkyl, wherein the alkyl can be linear, branched or cyclic, saturated or unsaturated,
      vii) keto,
      viii) nitro,
      ix) a saturated or unsaturated cyclic moiety, an aromatic or a heterocyclic moiety; the cyclic moiety having from 5-20 atoms comprising one or two-ringed structures, wherein each ring comprises 3-8 carbons, optionally interrupted by 1-4 heteroatoms, and optionally further substituted with one or more groups selected from i)-viii) as defined above,
b) an amine or an amide substituted with at least one substituent as defined in R′ above,
c) a thiol, a sulfide, a sulfoxide, a sulfone, a thioester or a thioamide optionally substituted with one substituent as defined in R′ above, and
d) a hydroxyl or an ether —OR′ wherein R′ is as defined above;
   R₂ is selected from the group consisting of
a) a halogen,
b) a linear, branched or cyclic, saturated or unsaturated C₁-C₆ alkyl, and
c) —OR wherein R is selected from the group consisting of
   A) -R″, wherein R″ is hydrogen or a linear, branched or cyclic, saturated or unsaturated C₁-C₆ alkyl optionally containing a terminal —OR′″ or —OC(O)R′″ moiety wherein R′″ is hydrogen or a linear, branched or cyclic, saturated or unsaturated C₁-C₆ alkyl, and
   B) —C(O)R′″ wherein R′″ is as previously defined; and
      R₃ is selected from the group consisting of
a) a linear, branched or cyclic, saturated or unsaturated C₁-C₁₂ alkyl,
b) —OR^{a}, in which R^{a} is a linear, branched or cyclic, saturated or unsaturated C₂-C₉ alkyl which may be substituted at the terminal carbon atom by a phenyl group, and
c) a linear, branched or cyclic, saturated or unsaturated C₁-C₇ alkyl-OR′″ wherein R′″ is as previously defined;
   and pharmaceutically acceptable salts, esters or solvates thereof

The present invention also encompasses pharmaceutical composition for increasing the transcription of at least one of the anti-inflammatory cytokine IL-10, the protective cytokine IL-6 and of the suppressors of cytokine signaling SOCS-1 and SOCS-3, comprising as an active ingredient a compound of general formula (I) as previously defined.

Currently more preferred compounds are those wherein R₂ is hydroxy or lower acyloxy and wherein R₃ is dimethylheptyl or a dimethylalkyl radical with a total of at least 7 carbon atoms.

According to currently preferred embodiments of the present invention R₁ is a heterocyclic moiety selected from the group consisting of an imidazolyl, an imidazolinyl, a morpholino, a piperidyl, a piperazinyl, a pyrazolyl, a pyrrolyl, a pyrrolidinyl, a triazolyl, and a tetrazolyl, wherein each cyclic moiety may optionally be further substituted with at least one substituent selected from the group consisting of C₁₋₆ alkyl, C₁₋₆ alkyloxy, C₁₋₆ alkylthio, keto, carboxy, nitro, saturated or unsaturated cyclic moieties or aromatic or heterocyclic moieties wherein each ring comprises 3-8 carbons optionally interrupted by 1-4 heteroatoms, said heteroatoms each independently selected from the group consisting of N, O, and S, wherein each ring optionally is further substituted with one or more groups selected from the group consisting of C₁₋₆ alkyl, C₁₋₆ alkyloxy, C₁₋₆ alkylthio, keto, carboxy, or nitro, wherein C₁₋₆ alkyl, C₁₋₆ alkoxy and C₁₋₆ alkylthio are intended to include saturated and unsaturated linear, branched and cyclic structures.

According to more preferred embodiments of the present invention R₁ is selected from the group consisting of hydroxyl, imidazole, pyrazole, oxazole, isoxazole, tetrahydropyridine, pyrazoline, oxazoline, pyrrolidine, imidazoline, 2-thio-imidazole, 2-methylthio-imidazoline, 4-methyl-2-imidazoline, 4,4-dimethyl-2-imidazoline, methyl sulfide, methylsulfoxide, acetamido, benzamide, cyano, 1,2,4-triazole, 1,3,4-triazole, 1,2,3,4-tetrazole, 1,2,3,5-tetrazole, thiophene, phenyl, morpholine, thiomorpholine, thiazolidine, glycerol, piperazine, 4-piperidinopiperidine, 4-methylpiperidine and tetrahydropyran.

According to additional more preferred embodiments of the present invention R₁ is selected from the group consisting of mono or di-substituted amines wherein the substituent is selected from the group consisting of an C₁₋₆ alkyl, C₁₋₆ alkyloxy, C₁₋₆ alkylthio, imidazolyl, an imidazolinyl, a morpholino, a piperidyl, a piperazinyl, a pyrazolyl, a pyrrolyl, a pyrrolidinyl, a triazolyl, and a tetrazolyl, wherein each cyclic moiety may optionally be further substituted with at least one substituent selected from the group consisting of C₁₋₆ alkyl, C₁₋₆ alkyloxy, C₁₋₆ alkylthio, keto, carboxy, nitro, saturated or unsaturated cyclic moieties or aromatic or heterocyclic moieties wherein each ring comprises 3-8 carbons optionally interrupted by 1-4 heteroatoms, said heteroatoms each independently selected from the group consisting of N, O, and S, wherein each ring optionally is further substituted with one or more groups selected from the group consisting of C₁₋₆ alkyl, C₁₋₆ alkyloxy, C₁₋₆ alkylthio, keto, carboxy, or nitro, wherein C₁₋₆ alkyl, C₁₋₆ alkoxy and C₁₋₆ alkylthio are intended to include saturated and unsaturated linear, branched and cyclic structures.

According to another currently preferred embodiment, we disclose a pharmaceutical composition which down-regulates gene expression of at least one the pro-inflammatory mediators COX-2, IL-1β, IL-2, iNOS, TNF-α and MCP-1, and up-regulates gene expression of at least one of the anti-inflammatory cytokine IL-10, the protective cytokine IL-6 and of the suppressors of cytokine signaling SOCS-1 and SOCS-3, comprising as an active ingredient a compound of the general formula (I) wherein R₂ is OH, R₃ is 1,1-dimethylheptyl, there is a double bond between C6 and C1, and R₁ is selected from the group consisting of hydroxyl, 2-mercaptoimidazole, imidazole, pyrazole, 4-methyl-piperidine, and 4-piperidino-piperidine.

The pharmaceutical compositions may contain in addition to the active ingredient conventional pharmaceutically acceptable carriers, diluents and excipients necessary to produce a physiologically acceptable and stable formulation.

The pharmaceutical compositions can be administered by any conventional and appropriate route including oral, parenteral, intravenous, intramuscular, intralesional, subcutaneous, transdermal, intrathecal, rectal or intranasal.

Prior to their use as medicaments for preventing, alleviating or treating an individual in need thereof, the pharmaceutical compositions will be formulated in unit dosage. The selected dosage of active ingredient depends upon the desired therapeutic effect, the route of administration and the duration of treatment desired.

A further aspect of the present invention provides a method of preventing, alleviating or treating a patient by regulating pro- and anti-inflammatory mediators selected from COX-2, IL-1β, IL-2, iNOS, TNF-α, MCP-1, IL-10, IL-6, SOCS-1 and SOCS-3, by administering to said patient a therapeutically effective amount of pharmaceutical composition containing as an active ingredient a compound of general formula (I) as previously defined.

A further aspect of the present invention relates to the use for the manufacture of a medicament for preventing, alleviating or treating a disease by regulating pro- and anti-inflammatory mediators selected from COX-2, IL-1β, IL-2, iNOS, TNF-α, MCP-1, IL-10, IL-6, SOCS-1 and SOCS-3, of a compound of general formula (I) substantially as shown in the specification.

### BRIEF DESCRIPTION OF THE FIGURES

To assist in the understanding of the invention, and in particular of the data that are given in the examples, the following drawing figures are presented herein:

FIG. 1 shows the effect of various doses of dexanabinol and its analogs on IL-2 in Jurkat cells activated with PMA and Calcium ionophore. In panel A the down-regulatory effect is measured at the level of IL-2 gene expression by real-time RT-PCR. In panel B the down-regulatory effect is measured at the level of IL-2 secretion.

FIG. 2 shows the down-regulatory effect of 10 μM of dexanabinol and its analogs on COX-2 gene expression in Jurkat cells activated with PMA and Calcium ionophore, as measured by real-time RT-PCR.

FIG. 3 shows the effect of dexanabinol and its analogs on gene expression in the brains of mice submitted to MCAo 18 hrs before the measurements. In panel A the down-regulatory effect is measured on the pro-inflammatory mediators COX-2, MCP-1 and IL-2. In panel B the up-regulatory impact is measured on anti-inflammatory IL-10.

FIG. 4 shows the effect of PRS-211,092 (▴) as compared to vehicle (▪) on expression of various inflammatory related genes as a function of time from ConA induction of liver injury. A: IL-2; B: MCP-1; C: TNF-α; D: IL-1β; E: IL-6; F: SOCS-1; and G: SOCS-3.

FIG. 5 shows the effect of various doses of PRS-211,092 and PRS-211,220 on NF-AT driven expression of luciferase in activated T cells.

FIG. 6 shows the effect of various doses of dexanabinol, PRS-211,092 and PRS-211,220, as well as Celecoxib and Dexamethasone (DXM), as compared to vehicle, on paw thickness in carrageenan induced paw edema.

FIG. 7 shows the effect of various doses of dexanabinol on tumor growth of in vivo implanted LoVo colorectal cancer cells.

FIG. 8 shows the effect of dexanabinol and PRS-211,220 in vivo in the MPTP model of neurodegeneration. In panel A the neurological outcome is measured in a short-term study. In panel B the functional outcome is measured using the rotarod test in a long-term study.

### DETAILED DESCRIPTION OF THE INVENTION

The present invention provides compositions effective to cause a reduction in gene expression of at least one of COX-2, IL-1β, IL-2, iNOS, TNF-α and MCP-1. Moreover, compositions of the present invention cause an increase in gene expression of at least one of the anti-inflammatory cytokine IL-10, the protective cytokine IL-6 and of the suppressors of cytokine signaling SOCS-1 and SOCS-3. The mechanism of action can either be through direct regulation of gene expression or through indirect feedback mechanisms. It will be noted that the compounds of the present invention have been tested for their impact on gene expression on a limited set of genes selected for their known involvement in the immunomodulatory and/or anti-inflammatory signaling cascades. Assaying the effect of those non-psychotropic cannabinoid derivatives on a larger set of genes, such as found in microarrays, may reveal additional genes that are involved in the new gene regulatory action herein disclosed.

The present invention relates to THC-type compounds which are characterized by an absolute stereochemistry at the positions 3 and 4 of the molecule (3S,4S), which is opposite to the (3R,4R) configuration in the natural series. The natural compounds of the (3R,4R) configuration produce undesirable psychotropic “cannabis” type effects, which preclude their use for other therapeutically interesting effects. The compounds of the invention being of the (3S,4S) configuration are substantially devoid of the undesired psychotropic effect and thus can be used for the treatment of various diseases and disorders. Thus, in the present specification and claims which follow the term “essentially free” qualitatively refer to (3S,4S) compounds of high optical purity substantially devoid of the undesired psychotropic effect lying with the (3R,4R) enantiomer. The quantitative criterion of the minimum acceptable degree of optical purity of an intended therapeutic enantiomer is dictated by the pharmacological potency of the opposite enantiomer. The higher the psychotropic activity of the opposite enantiomer, the stricter is the requirement for optical purity. Therefore, the quantitative meaning of “essentially free” depends upon the nature of each enantiomeric pair. The enantiomeric pair HU-210 and HU-211, respectively of (3R,4R) and (3S,4S) configuration, is an extreme example of such a situation, HU-210 being hundred times more psychoactive than natural Δ⁹-THC, the major active constituent in marijuana. The very highly undesirable psychotropic effects of HU-210 require that HU-211 should be of very high enantiomeric purity of at least 99.8% (Mechoulam R. et al., Tetrahedron Asymmetry 1(5): 315-8, 1990).

In the present specification the term “prodrug” represents compounds which are rapidly transformed in vivo to the parent compounds of formula (I), for example by hydrolysis in blood. Some of the compounds of formula (I) are capable of further forming pharmaceutically acceptable salts and esters. “Pharmaceutically acceptable salts and esters” means any salt and ester that is pharmaceutically acceptable and has the desired pharmacological properties. Such salts include salts that may be derived from an inorganic or organic acid, or an inorganic or organic base, including amino acids, which is not toxic or undesirable in any way. The present invention also includes within its scope solvates of compounds of formula (I) and salts thereof, for example, hydrates. All of these pharmaceutical forms are intended to be included within the scope of the present invention.

In the present specification “inhibiting, reducing, or decreasing effect” is the ability to reduce the activity under discussion by at least 20%, preferably 40%, more preferably 60% and most preferably 80% or greater.

In the present specification “enhancing or increasing effect” is the ability to increase the activity under discussion by at least 2 folds, preferably 3 folds, more preferably 4 folds and most preferably 5 folds or more.

In the present specification and claims which follow “prophylactically effective” is intended to qualify the amount of compound which will achieve the goal of prevention, reduction or eradication of the risk of occurrence of the disorder, while avoiding adverse side effects. The term “therapeutically effective” is intended to qualify the amount of compound that will achieve, with no adverse effects, alleviation, diminished progression or treatment of the disorder, once the disorder cannot be further delayed and the patients are no longer asymptomatic. The compositions of the present invention are prophylactic as well as therapeutic.

The “individual” or “patient” for purposes of treatment includes any human or mammalian subject affected by any of the diseases where the treatment has beneficial therapeutic impact.

Throughout this specification and the claims which follow, the alkyl substituents can be saturated or unsaturated, linear branched or cyclic, the latter only when the number of carbon atoms in the alkyl chain is equal or superior to 3.

The genes affected by compounds of the invention are listed below and the diseases wherein abnormal regulation of said genes is implicated in the pathological progression are briefly reviewed.

COX-2

In addition to their specific physiological functions in several organs, the kidney and gastrointestinal (GI) system in particular, prostaglandins have been known for some time to play a major role in the inflammatory process. They are involved as mediators of pain, edema and vascular permeability in arthritic diseases and they have been postulated to be involved in the pathophysiology of colorectal cancer. The biosynthesis of prostaglandins depends upon the action of cyclooxygenase (COX), recently found to exist in the human as cyclooxygenase type 1 (COX-1) and cyclooxygenase type 2 (COX-2). Both enzymes are involved in the synthesis of prostaglandins, COX-1 constitutively and COX-2 following induction by a number of agents including mitogens, endotoxins, hormones, cytokines, stress conditions and growth-factors. As prostaglandins have both physiological and pathological roles, it has been assumed that the constitutive COX-1 was responsible for the important physiological functions for example in the GI tract, while the inducible COX-2 was mainly responsible for the pathological effects of prostaglandins in inflamed tissues. However, recent pharmacological studies have shown that COX-2 is not exclusively expressed in inflamed tissues, but is constitutively present in several organs where it synergizes with COX-1 in maintaining homeostasis. Therefore, total inhibition of COX-2 might not be desirable. Nevertheless, a selective inhibitor of COX-2 is expected to be useful in treating the pathophysiological effects of prostaglandins, by virtue of its anti-inflammatory, antipyretic and analgesic properties. Indeed COX-2 inhibitors already exist on the market and have a wide range of therapeutic benefits. COX-2 inhibitors were already found effective in the treatment of osteoarthritis, rheumatoid arthritis, ocular inflammation, acute and chronic menstrual pain, gastritis caused by bacterium helicobacter pylori. In addition a selective inhibitor would have potential anti-cancer effects, such as with breast and colorectal cancer, would be useful in the treatment of polyps and angiogenesis and be an attractive candidate for the treatment of neurological damage either resulting from spinal cord injury, cerebral ischemia or neurodegenerative disorders such as Alzheimer disease and Parkinson's disease or AIDS associated dementia A positive role for COX-2 inhibition has also been suggested in chronic liver diseases such as cirrhosis. Recent finding suggest that COX-2 is a major source of systemic prostacyclin synthesis, and its increased production is observed in patients with signs of platelet activation such as unstable angina, severe atherosclerosis and during angioplasty.

IL-1β

Interleukin-1β (IL-1β) is a potent pro-inflammatory cytokine that has been implicated in a broad spectrum of diseases. Cells known to express IL-1β include astrocytes, adrenal cortical cells, NK cells, macrophages and monocytes, endothelial cells, keratinocytes, megakaryocytes and platelets, neurons, neutrophils, oligodendroglia, osteoblasts, Schwann cells, trophoblasts, and T cells plus fibroblasts. IL-1β is a key factor in several inflammatory disorders that accompany for example septic shock, IBD, pancreatitis, ulcerative colitis, pulmonary inflammation and wound healing. It is also implicated in inflammation leading to connective tissue destruction such as in rheumatoid arthritis, osteoarthritis, synovial inflammation and periodontal disease. Moreover, it is involved in central nervous system (CNS) pathologies, where it is thought to exacerbate neuronal loss. Its level is elevated in brain injury, ALS, AD, PD and anorexia. However, it remains unclear how the effects of IL-1β are mediated. Exacerbation may be a result of either the direct cytotoxic action of IL-1β on resident cells in the CNS or it may be a result of secondary bystander damage by the leukocytes recruited to the brain in response to IL-1β production. Inappropriate production of IL-1β was also observed in immune disorders such as allergy, systemic lupus erythematosus (SLE), psoriasis, graft versus host disease and MS. IL-1β is up-regulated in patients suffering from Gaucher's disease (GD), and it is speculated that this over-expression may relate to the pathophysiology of some of the clinical manifestations of GD. The promotion of pancreatic beta-cell destruction leading to insulin dependent diabetes mellitus shows dependence on IL-1. The acquired expression of IL-1β, which might affect the production of other various cytokines as well as the regulation of other cellular factors, has been implicated in the progression of benign oncologic conditions to severe and often fatal malignancies, in atherosclerosis and other cardiovascular disorders, in infectious diseases, in renal and liver dysfunction, acute respiratory distress syndrome (ARDS), in ischemic and reperfusion injury and multiple organ failure. The long list of diseases wherein IL-1β was found implicated supports its central role as a pivotal pro-inflammatory mediator.

IL-2

The induction of the IL-2 gene is a key event in T cell activation that is required for the resting cells to become effector cells. Following ligand binding to the T cell receptor together with the engagement of a co-stimulatory receptor such as CD28, a cascade of cytoplasmic signaling molecules leads to the assembly of several transcription factors at their corresponding sites on the IL-2 promoter region in the nucleus. Each one of these transcription factors, including AP-1, NF-κB and NFAT, is regulated by different signaling pathways, which act in concert to elicit full activation of the IL-2 gene. Similar mode of IL-2 gene regulation is obtained by T cell activation with phorbol myristate acetate (PMA) and calcium ionophore. Immunosuppressive drugs such as cyclosporin A strongly inhibit the transcription of the IL-2 gene and their contribution to the therapeutic arsenal is well known. Compounds able to inhibit the production of the cytokine IL-2 are potential immunosuppressive drugs important in the treatment of disorders where T cell activation plays a pivotal role, in particular inflammatory conditions with an etiology including an autoimmune component such as arthritis, rheumatic diseases, systemic lupus erythematosus, myasthenia gravis, inflammatory bowel disease, chronic liver disease, heart failure, multiple sclerosis, inflammatory demyelinating neuropathies, psoriasis, diabetes type 1, parasitic infections, uveitis and other ocular inflammatory conditions, Sjögren's syndrome, glomerulonephritis and transplant rejection.

IL-6

Interleukin-6 (IL-6) is critical to the regulation of the immune and haematopoietic systems. The pleiotropic nature of this cytokine family has resulted in a variety of pseudonames based on its multiple biological functions. IL-6 elicits B cells to undergo proliferation and differentiation into antibody-forming cells; assists in IL-4 dependant IgE synthesis and T cell activation, growth and differentiation. IL-6 also acts in conjunction with IL-3 to induce the proliferation of pluripotent haematopoietic progenitors. In addition, this cytokine induces the expression of acute phase proteins. IL-6 is secreted by T cells, B cells, mast cells, monocytes, macrophages, hepatocytes, fibroblasts, endothelial cells, keratinocytes and many tumor cell lines. Adipocytes, bone marrow stroma cells, mesangial cells and some cell types of the central nervous system also produce this cytokine. IL-6 production is generally correlated with cell activation. IL-6 has been described as both a pro-inflammatory and anti-inflammatory mediator, and its levels are altered in a number of diseases: in inflammatory and autoimmune diseases, such as RA and other forms of arthritis, SLE, ulcerative colitis, Crohn's disease, pancreatitis, diabetes, MS, psoriasis; in infectious diseases, such as HIV, bacterial infections and sepsis, viral and bacterial meningitis; in oncologic disorders such as metastatic melanoma, cervical cancer, myeloid leukemia, multiple myeloma, Hodgkin's disease, metastatic renal cell carcinoma, prostate tumors; in renal insufficiency and dialysis, such as glomerulonephritis, nephropaties and hemodialysis; in liver diseases, such as chronic liver diseases, alcoholic liver cirrhosis, hepatitis and hepatectomies; in kidney, bone marrow or liver transplantation; in AD, burns victims and patients suffering from myocardial infarct. IL-6 is considered to be an early marker of injury severity following trauma. The nature of IL-6 action depends upon time and site of expression, which ultimately influence if this cytokine will act as pro- or anti-inflammatory. However, recent studies suggest that IL-6 should be considered as a protective cytokine in the overall balance of cytokine regulation. Proper regulation of this important cytokine will have clear beneficial therapeutic impact.

IL-10

Interleukin-10 (IL-10) down-regulates the production of pro-inflammatory cytokines and chemokines by activated macrophages, monocytes, polymorphonuclear leukocytes and eosinophils. It has been recently suggested that part of this down-regulation is achieved by elevation in the levels of SOCS molecules (Suppressors Of Cytokine Signaling). Therefore IL-10 is an anti-inflammatory cytokine that plays a role in suppressing immune and inflammatory responses. There is evidence that IL-10 can control both T helper 1 (Th1) type of responses and also Th2 mediated inflammatory processes. IL-10 has a beneficial effect on a variety of acute and chronic inflammatory and autoimmune events including but not limited to rheumatoid arthritis, ischemia-reperfusion injury, atherosclerosis, psoriasis, pemphigus, allergic contact sensitivity reactions, uveitis, organ transplantation, injury, infection and sepsis, inflammatory bowel disease, acute pancreatitis, asthma, nephrotoxic nephritis and certain malignancies.

iNOS

Nitric oxide (NO) is a short lived molecule required for many physiological functions in host defence, inflammation and immunity. NO is synthesized by the enzyme nitric oxide synthase (NOS) and overproduced during various pathological inflammatory states. Three distinct isoforms of NOS have been identified: neuronal (nNOS), entothelial (eNOS) and inducible (iNOS). Overproduction of NO by nNOS and iNOS have been reported in a number of clinical disorders including acute and chronic neurodegenerative disorders, convulsions, pain, septic shock, asthma, tissue damage following inflammation, Crohn's disease, SLE, osteoarthritis, rheumatoid arthritis, allograft rejection and in certain cancers. iNOS is induced by endotoxin or pro-inflammatory cytokines and in turn modulate expression of the latter. Selective inhibition of nNOS or iNOS is expected to provide a novel therapeutic approach to various diseases.

MCP-1

Monocyte chemoattractant protein-1 (MCP-1) is a pro-inflammatory chemokine of the C—C family, responsible for the recruitment and activation of mainly monocytes, macrophages, basophils, mast cells, T cells, and natural killer cells. The activated monocytes, which are recruited to the site of injury, secrete in turn inflammatory agents such as TNF-α, IL-1β, nitric oxide and prostaglandins. MCP-1 can be involved in beneficial processes such as wound healing, but when expressed in excess it becomes involved in the pathophysiology of a large number of inflammatory and autoimmune diseases. MCP-1 has been implicated in a large number of diseases that affect various organs by means of acute or chronic inflammation. Pathological sites of action of MCP-1 include the skeleton with disorders such as rheumatoid arthritis and various types of osseous inflammation, the kidneys with nephrites, nephritic syndromes and nephrosis characterized by glomerular nephritides, the eyes with uveitis, vitreoretinal disorders, proliferative diabetic retinopathy, and other ocular inflammatory conditions, the cardiovascular system with MCP-1 involvement in the early stages of atherosclerosis, in restenosis and in the inflammatory response following myocardial infarction, the respiratory system with alveolitis, asthma and lung fibrosis or allergic inflammation, the digestive tract with inflammatory bowel diseases (IBD) including both ulcerative colitis and Crohn's disease. The nervous system is also an important target where MCP-1 up-regulation has been observed in various types of pathology. In the central nervous system, increases in MCP-1 level have been observed following both head trauma and brain ischemia as in stroke, and in immune mediated inflammation as seen in experimental autoimmune encephalomyelitis (EAE) or multiple sclerosis (MS), and in the inflammatory phenomenon associated with neurodegenerative disorders such as Alzheimer's disease (AD). Likewise, MCP-1 is also involved in the development of peripheral nervous system (PNS) disorders characterized by mononuclear cell infiltration and related demyelinating disorders. Moreover MCP-1 is involved in vasculitis, angiogenesis, tumor growth and metastasis, graft rejection, certain types of bacterial, parasitic or viral infections, psoriasis, pemphigus and related disorders, delayed type hypersensitivity reactions of the skin, Hodgkin's disease and a number of chronic diseases characterized by a significant infiltrate of monocytes, including sarcoidosis, Wegener's granulomatosis and tuberculosis. MCP-1 is also very important in cases where complications occur following surgical interventions such as, for example, angioplasty, atherectomy, circulatory recovery techniques, transplants, organ replacement, tissue replacement and prosthetic implants. Thus, blocking MCP-1 production and therefore leukocyte recruitment to target tissues in inflammatory and autoimmune disease would be a highly effective intervention.

SOCS

Cytokines exert their biological activity through binding to specific cell surface receptors, that initiate the appropriate intracellular signal transduction cascade which in turn lead to the physiological outcome. Despite their diverse biological roles, many common themes have emerged in cytokine signal transduction. Upon receptor multimerisation, the Janus kinases (JAKs) are activated and phosphorylate, among other proteins, the signal transducers and activators of transcription (STATs). Dimers of phosphorylated STATs then move into the nucleus where they bind to recognition sequence in target genes to increase transcription. Such signaling pathway needs to be also negatively regulated to ensure the timely switch off of the biological response. There are at least three families of proteins that inhibit JAK/STAT signaling and the suppressors of cytokine signaling (SOCS) belong to one of them. SOCS proteins are an important element in a classic negative feedback loop that regulates JAK/STAT signal transduction initiated by many cytokines. Following cytokine activation the cell will not only display increased transcription of the genes important in mediating the biological effects of the cytokine but also genes encoding the SOCS protein which limit the biological effect of the cytokine. At least seven SOCS proteins were identified till now and their activity is presently being unraveled. SOCS-1 and SOCS-3 are induced by about the same set of cytokines, including IL-6, Growth Hormone (GH), IL-10 and GM-CSF, though with different efficiency. In turn SOCS-1 inhibits IL-6, GH signaling, IL-2, IL-4, Interferons (IFN) α, β and γ, LIF, oncostatin M and trombopoeitin. SOCS-3 down-regulates leptin, GH, IFN-α, IFN-β and IFN-γ. Thus, induction of SOCS proteins by one cytokine also attenuates the response for additional cytokines different from the original inducer, by a phenomenon of cross inhibition. These proteins are potentially important regulators of inflammatory and immune responses of hematopoiesis and hormone response.

TNF-α

Tumor necrosis factor (TNF) has been attributed a central role in inflammatory processes. In all disorders with an inflammatory component the common process is that the initial injury, whether initiated by an infectious, chemical, or environmental agent, produces focal tissue necrosis in the target organ. As a result of this damage, tissue-fixed macrophages and circulating monocytes migrate to the damaged site, become activated and secrete products that cause additional cell damage or induction of inflammatory products thus amplifying the response. TNF-α is a cytokine produced primarily by monocytes and macrophages. Beside its physiologic effect, TNF-α is cytotoxic and regulates inflammatory processes through induction for instance of IL-1, IL-6, IL-8, macrophage inflammatory protein (MIP)-2, granulocyte-macrophage colony stimulating factor (GM-CSF) and adhesion molecules. At elevated levels TNF-α is involved in septic shock syndrome, autoimmune and inflammatory processes including Crohn's disease, brain injury, venous thromboses, arteriosclerosis, vasculitis, IBD, MS, EAE, SLE, AD, PD, AIDS dementia, contact dermatitis, mixed connective tissue disease, arthritis, organ specific toxic response, hepatic injury during sepsis and reperfusion, chronic inflammatory lung diseases, muscle wasting and cachexia. The therapeutic importance of TNF-α blockade is tremendous, nevertheless it should be kept in mind that TNF-α is also involved in normal physiological and repair processes and totally eliminating this cytokine would be detrimental.

By virtue of their anti-inflammatory and immunomodulatory properties, resulting from the gene regulation of the pro- and anti-inflammatory mediators selected COX-2, IL-1β, IL-2, iNOS, TNF-α, MCP-1, IL-10, IL-6, SOCS-1 and SOCS-3, it will be recognized that the compositions according to the present invention will be useful for treating indications having an inflammatory or autoimmune mechanism involved in their etiology or pathogenesis. Such diseases or disorders are exemplified by multiple sclerosis, amyotrophic lateral sclerosis, systemic lupus erythematosis, myasthenia gravis, Sjögren's syndrome, diabetes mellitus type I, late onset diabetes type 2, sarcoidosis; skeletal and connective tissue disorders including arthritis, rheumatoid arthritis, osteoarthritis and rheumatoid diseases; ocular inflammation related disorders including uveitis, vitreoretinal disorders, proliferative diabetic retinopathy, allergic conjunctivitis; metabolic diseases that involve abnormalities in lipid peroxisomes and lipid peroxidation and/or oxidative stress; skin related disorders including psoriasis, pemphigus and related syndromes, delayed-type hypersensitivity and contact dermatitis; respiratory diseases including cystic fibrosis, chronic bronchitis, emphysema, chronic obstructive pulmonary disease, asthma, allergic rhinitis or lung inflammation, idiopathic lung fibrosis, tuberculosis, and alveolitis; kidney diseases including autoimmune nephritis, renal ischemia, nephrites, nephritic syndromes and nephrosis characterized by glomerular nephritides; liver diseases both acute and chronic such as autoimmune hepatitis, cirrhosis, hepatitis and fulminant hepatic failure; gastrointestinal diseases including inflammatory bowel diseases, ulcerative colitis, Crohn's disease and gastritis, polyposis and cancer of the bowel, especially the colon; infectious diseases generated by certain bacterial, viral and parasitic invasion and sepsis that might result from injury; and post-operative complications following angioplasty, circulatory recovery techniques, prosthetic implants and tissue or organ transplants, including graft rejection.

When the site of action is the central or the peripheral nervous system, the pharmaceutical compositions comprising as an active ingredient a compound regulating the gene expression of the pro- and anti-inflammatory mediators selected from COX-2, IL-1β, IL-2, iNOS, TNF-α, MCP-1, IL-10, IL-6, SOCS-1 and SOCS-3, act as neuroprotectors. By virtue of their neuroprotective properties, it will be recognized that the compositions according to the present invention will be useful in treating acute neurological disorders, resulting either from ischemic or traumatic damage, including but not limited to stroke, head trauma and spinal cord injury. The composition of the present invention may also be effective in treating demyelinating disorders and certain chronic degenerative diseases that are characterized by gradual selective neuronal loss such as Parkinson's disease, Alzheimer's disease, AIDS dementia, Huntington's chorea, amyotrophic lateral sclerosis, Kennedy's syndrome, motor neuron disease and prion-associated neurodegeneration.

By virtue of their analgesic properties it will be recognized that the compositions according to the present invention will be useful in treating pain including peripheral, visceral, neuropathic, inflammatory and referred pain.

The compositions of the present invention may also be effective in cardiovascular protection and/or treatment of atheroma, atherosclerosis, and consequences thereof, restenosis, angioplasty, myocardial ischemia and myocardial infarction.

Another feature of the present invention is the ability of the disclosed compounds in chemoprevention in oncological processes including polyps, tumor growth, angiogenesis and metastasis of certain types of cancer, including breast and colon cancer.

The pharmaceutical compositions of the present invention, which down-regulate gene expression of at least one the pro-inflammatory mediators COX-2, IL-1β, IL-2, iNOS, TNF-α, and MCP-1, comprise as an active ingredient a compound of the general formula (I): 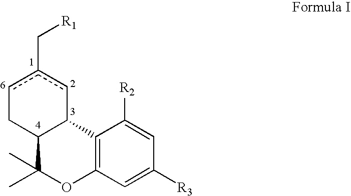
having the (3S,4S) configuration and being essentially free of the (3R,4R) enantiomer, wherein the dashed line indicates an optional C1-C2 or C6-C1 double bond, and wherein:
R₁ is selected from the group consisting of
a) R′ where R′ is selected from the group consisting of
   A) a linear or branched, saturated or unsaturated, carbon side chain comprising 1-8 carbon atoms optionally interrupted by 1-3 heteroatoms, and
   B) a saturated or unsaturated cyclic moiety, an aromatic moiety or a heterocyclic moiety; the cyclic moiety having from 5-20 atoms comprising one or two-ringed structures, wherein each ring comprises 3-8 carbons, optionally interrupted by 1-4 heteroatoms, and optionally further substituted with one or more groups selected from
      i) a linear, branched or cyclic, saturated or unsaturated C₁-C₆ alkyl,
      ii) a linear, branched or cyclic, saturated or unsaturated C₁-C₆ alkoxy,
      iii) a linear, branched or cyclic, saturated or unsaturated C₁-C₆ alkylthio,
      iv) a halogen,
      v) carboxyl,
      vi) —CO₂—C₁-C₄ alkyl, wherein the alkyl can be linear, branched or cyclic, saturated or unsaturated,
      vii) keto,
      viii) nitro,
      ix) a saturated or unsaturated cyclic moiety, an aromatic or a heterocyclic moiety; the cyclic moiety having from 5-20 atoms comprising one or two-ringed structures, wherein each ring comprises 3-8 carbons, optionally interrupted by 1-4 heteroatoms, and optionally further substituted with one or more groups selected from i)-viii) as defined above,
b) an amine or an amide substituted with at least one substituent as defined in R′ above,
c) a thiol, a sulfide, a sulfoxide, a sulfone, a thioester or a thioamide optionally substituted with one substituent as defined in R′ above, and
d) a hydroxyl or an ether —OR′ wherein R′ is as defined above;
   R₂ is selected from the group consisting of
a) a halogen,
b) a linear, branched or cyclic, saturated or unsaturated C₁-C₆ alkyl, and
c) —OR wherein R is selected from the group consisting of
   A) -R″, wherein R″ is hydrogen or a linear, branched or cyclic, saturated or unsaturated C₁-C₆ alkyl optionally containing a terminal —OR′″ or —OC(O)R′″ moiety wherein R′″ is hydrogen or a linear, branched or cyclic, saturated or unsaturated C₁-C₆ alkyl, and
   B) —C(O)R′″ wherein R′″ is as previously defined; and
      R₃ is selected from the group consisting of
a) a linear, branched or cyclic, saturated or unsaturated C₁-C₁₂ alkyl,
b) —OR^{a}, in which R^{a} is a linear, branched or cyclic, saturated or unsaturated C₂-C₉ alkyl which may be substituted at the terminal carbon atom by a phenyl group, and
c) a linear, branched or cyclic, saturated or unsaturated C₁-C₇ alkyl-OR′″ wherein R′″ is as previously defined;
   and pharmaceutically acceptable salts, esters or solvates thereof.

The pharmaceutical compositions of the present invention, which up-regulate gene expression of at least one of the anti-inflammatory cytokine IL-10, the protective cytokine IL-6 and of the suppressors of cytokine signaling SOCS-1 and SOCS-3, comprise as an active ingredient a compound of the general formula (I): 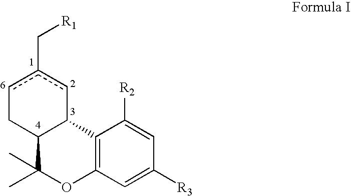
having the (3S,4S) configuration and being essentially free of the (3R,4R) enantiomer, wherein the dashed line indicates an optional C1-C2 or C6-C1 double bond, and wherein:
R₁ is selected from the group consisting of
a) R′ where R′ is selected from the group consisting of
   A) a linear or branched, saturated or unsaturated, carbon side chain comprising 1-8 carbon atoms optionally interrupted by 1-3 heteroatoms, and
   B) a saturated or unsaturated cyclic moiety, an aromatic moiety or a heterocyclic moiety; the cyclic moiety having from 5-20 atoms comprising one or two-ringed structures, wherein each ring comprises 3-8 carbons, optionally interrupted by 1-4 heteroatoms, and optionally further substituted with one or more groups selected from
      i) a linear, branched or cyclic, saturated or unsaturated C₁-C₆ alkyl,
      ii) a linear, branched or cyclic, saturated or unsaturated C₁-C₆ alkoxy,
      iii) a linear, branched or cyclic, saturated or unsaturated C₁-C₆ alkylthio,
      iv) a halogen,
      v) carboxyl,
      vi) —CO₂—C₁-C₄ alkyl, wherein the alkyl can be linear, branched or cyclic, saturated or unsaturated,
      vii) keto,
      viii) nitro,
      ix) a saturated or unsaturated cyclic moiety, an aromatic or a heterocyclic moiety; the cyclic moiety having from 5-20 atoms comprising one or two-ringed structures, wherein each ring comprises 3-8 carbons, optionally interrupted by 1-4 heteroatoms, and optionally further substituted with one or more groups selected from i)-viii) as defined above,
b) an amine or an amide substituted with at least one substituent as defined in R′ above,
c) a thiol, a sulfide, a sulfoxide, a sulfone, a thioester or a thioamide optionally substituted with one substituent as defined in R′ above, and
d) a hydroxyl or an ether —OR′ wherein R′ is as defined above;
   R₂ is selected from the group consisting of
a) a halogen,
b) a linear, branched or cyclic, saturated or unsaturated C₁-C₆ alkyl, and
c) —OR wherein R is selected from the group consisting of
   A) -R″, wherein R″ is hydrogen or a linear, branched or cyclic, saturated or unsaturated C₁-C₆ alkyl optionally containing a terminal —OR′″ or —OC(O)R′″ moiety wherein R′″ is hydrogen or a linear, branched or cyclic, saturated or unsaturated C₁-C₆ alkyl, and
   B) —C(O)R′″ wherein R′″ is as previously defined; and
      R₃ is selected from the group consisting of
a) a linear, branched or cyclic, saturated or unsaturated C₁-C₁₂ alkyl,
b) —OR^{a}, in which R^{a} is a linear, branched or cyclic, saturated or unsaturated C₂-C₉ alkyl which may be substituted at the terminal carbon atom by a phenyl group, and
c) a linear, branched or cyclic, saturated or unsaturated C₁-C₇ alkyl-OR′″ wherein R′″ is as previously defined;
   and pharmaceutically acceptable salts, esters or solvates thereof.

Currently more preferred compounds are those wherein R₂ is hydroxy or lower acyloxy and wherein R₃ is dimethylheptyl or a dimethylalkyl radical with a total of at least 7 carbon atoms.

According to currently preferred embodiments of the present invention R₁ is a heterocyclic moiety selected from the group consisting of an imidazolyl, an imidazolinyl, a morpholino, a piperidyl, a piperazinyl, a pyrazolyl, a pyrrolyl, a pyrrolidinyl, a triazolyl, and a tetrazolyl, wherein each cyclic moiety may optionally be further substituted with at least one substituent selected from the group consisting of C₁₋₆ alkyl, C₁₋₆ alkyloxy, C₁₋₆ alkylthio, keto, carboxy, nitro, saturated or unsaturated cyclic moieties or aromatic or heterocyclic moieties wherein each ring comprises 3-8 carbons optionally interrupted by 1-4 heteroatoms, said heteroatoms each independently selected from the group consisting of N, O, and S, wherein each ring optionally is further substituted with one or more groups selected from the group consisting of C₁₋₆ alkyl, C₁₋₆ alkyloxy, C₁₋₆ alkylthio, keto, carboxy, or nitro, wherein C₁₋₆ alkyl, C₁₋₆ alkoxy and C₁₋₆ alkylthio are intended to include saturated and unsaturated linear, branched and cyclic structures.

According to more preferred embodiments of the present invention R₁ is selected from the group consisting of hydroxyl, imidazole, pyrazole, oxazole, isoxazole, tetrahydropyridine, pyrazoline, oxazoline, pyrrolidine, imidazoline, 2-thio-imidazole, 2-methylthio-imidazoline, 4-methyl-2-imidazoline, 4,4-dimethyl-2-imidazoline, methyl sulfide, methylsulfoxide, acetamido, benzamide, cyano, 1,2,4-triazole, 1,3,4-triazole, 1,2,3,4-tetrazole, 1,2,3,5-tetrazole, thiophene, phenyl, morpholine, thiomorpholine, thiazolidine, glycerol, piperazine, 4-piperidinopiperidine, 4-methylpiperidine and tetrahydropyran.

According to additional more preferred embodiments of the present invention R₁ is selected from the group consisting of mono or di-substituted amines wherein the substituent is selected from the group consisting of an C₁₋₆ alkyl, C₁₋₆ alkyloxy, C₁₋₆ alkylthio, imidazolyl, an imidazolinyl, a morpholino, a piperidyl, a piperazinyl, a pyrazolyl, a pyrrolyl, a pyrrolidinyl, a triazolyl, and a tetrazolyl, wherein each cyclic moiety may optionally be further substituted with at least one substituent selected from the group consisting of C₁₋₆ alkyl, C₁₋₆ alkyloxy, C₁₋₆ alkylthio, keto, carboxy, nitro, saturated or unsaturated cyclic moieties or aromatic or heterocyclic moieties wherein each ring comprises 3-8 carbons optionally interrupted by 1-4 heteroatoms, said heteroatoms each independently selected from the group consisting of N, O, and S, wherein each ring optionally is further substituted with one or more groups selected from the group consisting of C₁₋₆ alkyl, C₁₋₆ alkyloxy, C₁₋₆ alkylthio, keto, carboxy, or nitro, wherein C₁₋₆ alkyl, C₁₋₆ alkoxy and C₁₋₆ alkylthio are intended to include saturated and unsaturated linear, branched and cyclic structures.

According to another currently preferred embodiment, we disclose a pharmaceutical composition which down-regulates gene expression of at least one the pro-inflammatory mediators COX-2, IL-1β, IL-2, iNOS, TNF-α and MCP-1, and up-regulates gene expression of at least one of the anti-inflammatory cytokine IL-10, the protective cytokine IL-6 and of the suppressors of cytokine signaling SOCS-1 and SOCS-3, comprising as an active ingredient a compound of the general formula (I) wherein R₂ is OH, R₃ is 1,1-dimethylheptyl, there is a double bond between C6 and C1, and R₁ is selected from the group consisting of hydroxyl, 2-mercaptoimidazole, imidazole, pyrazole, 4-methyl-piperidine, and 4-piperidino-piperidine.

Specific pharmaceutical compositions of particular interest comprise as an active ingredient compounds within formula (I) previously disclosed as HU-211, also known as dexanabinol, PRS-211,092, PRS-211,095, PRS-211,220, PRS-211,251 and PRS-211,257 in International Patent application WO 01/98289.

The novel compositions contain in addition to the active ingredient conventional pharmaceutically acceptable carriers, diluents and excipients necessary to produce a physiologically acceptable and stable formulation. Some compounds of the present invention are characteristically hydrophobic and practically insoluble in water with high lipophilicity, as expressed by their high octanol/water partition coefficient expressed as log P values, and formulation strategies to prepare acceptable dosage forms will be applied. Enabling therapeutically effective and convenient administration of the compounds of the present invention is an integral part of this invention.

For water soluble compounds standard formulations will be utilized. Solid compositions for oral administration such as tablets, pills, capsules, softgels or the like may be prepared by mixing the active ingredient with conventional, pharmaceutically acceptable ingredients such as corn starch, lactose, sucrose, mannitol, sorbitol, talc, polyvinylpyrrolidone, polyethyleneglycol, cyclodextrins, dextrans, glycerol, polyglycolized glycerides, tocopheryl polyethyleneglycol succinate, sodium lauryl sulfate, polyethoxylated castor oils, non-ionic surfactants, stearic acid, magnesium stearate, dicalcium phosphate and gums as pharmaceutically acceptable diluents. The tablets or pills can be coated or otherwise compounded with pharmaceutically acceptable materials known in the art, such as microcrystalline cellulose and cellulose derivatives such as hydroxypropylmethylcellulose (HPMC), to provide a dosage form affording prolonged action or sustained release. Other solid compositions can be prepared as suppositories, for rectal administration. Liquid forms may be prepared for oral administration or for injection, the term including but not limited to subcutaneous, transdermal, intravenous, intrathecal, intralesional, adjacent to or into tumors, and other parenteral routes of administration. The liquid compositions include aqueous solutions, with or without organic cosolvents, aqueous or oil suspensions including but not limited to cyclodextrins as suspending agent, flavored emulsions with edible oils, triglycerides and phospholipids, as well as elixirs and similar pharmaceutical vehicles. In addition, the compositions of the present invention may be formed as aerosols, for intranasal and like administration. Topical pharmaceutical compositions of the present invention may be formulated as solution, lotion, gel, cream, ointment, emulsion or adhesive film with pharmaceutically acceptable excipients including but not limited to propylene glycol, phospholipids, monoglycerides, diglycerides, triglycerides, polysorbates, surfactants, hydrogels, petrolatum or other such excipients as are known in the art.

Prior to their use as medicaments, the pharmaceutical compositions will generally be formulated in unit dosage. The active dose for humans is generally in the range of from 0.05 mg to about 50 mg per kg body weight, in a regimen of 1-4 times a day. The preferred range of dosage is from 0.1 mg to about 20 mg per kg body weight. However, it is evident to the man skilled in the art that dosages would be determined by the attending physician, according to the disease to be treated, its severity, the method and frequency of administration, the patient's age, weight, gender and medical condition, contraindications and the like. The dosage will generally be lower if the compounds are administered locally rather than systematically, and for prevention or chronic treatment rather than for acute therapy.

A further aspect of the present invention provides a method of preventing, alleviating or treating a patient by regulating pro- and anti-inflammatory mediators selected from COX-2, IL-1β, IL-2, iNOS, TNF-α, MCP-1, IL-10, IL-6, SOCS-1 and SOCS-3, by administering to said patient a therapeutically effective amount of pharmaceutical composition containing as an active ingredient a compound of general formula (I) as previously defined.

A further aspect of the present invention relates to the use for the manufacture of a medicament for preventing, alleviating or treating a disease by regulating pro- and anti-inflammatory mediators selected from COX-2, IL-1β, IL-2, iNOS, TNF-α, MCP-1, IL-10, IL-6, SOCS-1 and SOCS-3, of a compound of general formula (I) substantially as shown in the specification.

The principles of the present invention will be more fully understood in the following examples, which are to be construed in a non-limitative manner.

### PHYSIOLOGICAL EXAMPLES

Unless otherwise indicated the test compounds are prepared as follows: for in vitro assays the compounds are first dissolved in DMSO and then stepwise diluted in the assay buffer, generally tissue culture medium, down to a final concentration of 0.1% DMSO. For in vivo assays the test compounds are first diluted in cremophor:ethanol (70% and 30% w/w respectively) and further diluted 1:20 in physiological buffer, generally saline, to reach the appropriate dose. Alternatively, compounds can be first disolved in PEG:ethanol (1:1) and then diluted in Intralipid. Thus, the vehicle is the original “solvent” diluted in the appropriate buffer.

During this study, we concentrated our attention toward genes involved in inflammatory processes either directly, e.g. cytokines and chemokines, or indirectly, e.g. regulators of cytokine signaling pathways or transcription. These genes are either encoding pro-inflammatory mediators, such as the cytokines IL-1β, IL-2, TNF-α, the chemoline MCP-1 and the enzyme COX-2, or anti-inflammatory mediators such as the anti-inflammatory cytokine IL-10, the protective cytokine IL-6 or upstream regulators of cytokine such as the suppressors of cytokine signaling SOCS-1 and SOCS-3. Gene expression was assessed by RNA level quantitation and when possible by direct protein quantitation. Whatever the experimental system or the gene to be tested the following procedure were followed for quantitation.

A—Impact of Tricyclic Dextrocannabinoids on Gene Expression

### Example 1

### RNA Preparation and Real-time RT-PCR

Total RNA was prepared using SV total RNA isolation system (Promega). The cells or tissues were homogenized in lysis buffer. The lysates were transferred to an RNA isolation column, treated with DNAse, washed and eluted according to kit instructions. RNA concentrations were determined using GeneQuant II (Phamacia-Amersham). Complementary DNA (cDNA) was synthesized from total RNA using SUPERSCRIPT II reverse transcriptase (Life Technologies). 2 μg of total RNA were combined with an oligo (dT)₁₅ primer, 0.5 mM dNTP mix, 8 units of reverse transcriptase and other reaction components up to a final volume of 20 μl according to the kit instructions. The reaction mixture was incubated at 42° C. for 45 min and inactivated at 70° C. for 15 minutes. Quantitative real-time RT-PCR includes 1 μl of the cDNA, 300 nM of the appropriate forward and reverse primers (see below) and 7.5 μl of the reaction mix containing buffer, nucleotides, Taq polymerase and syber green (Syber Green master mix, Applied Biosystems), in a total reaction volume of 15 μl. Gene amplification was obtained using the GeneAmp 5700 sequence detection system (Applied Biosystems). Amplification included one stage of 10 minutes at 95° C. followed by 40 cycles of a 2-steps loop: 20 seconds at 95° C., and 1 minute at 60° C. During each annealing step, the amount of the amplified product is measured by the fluorescence of the double strand DNA binding dye, syber Green. The cycle of threshold (C_{T}), representing the PCR cycle at which an increase in fluorescence above a baseline signal can be first detected, is determined for each product. A delay of one PCR cycle in the C_{T} is translated into a two-fold decrease in starting template molecules and vice versa. The changes in the C_{T} of the specific gene product are normalized to the changes in the C_{T} of a reference gene cyclophilin A or GAPDH. Results are expressed as fold increase of gene expression in the test system above the appropriate control, such as inactivated cell lines or vehicle “treated” animals. In all cases, results are also normalized to either one of the reference house-keeping genes.

Primer sequences used:

| | |
|---|---|
| Mouse COX-2 forward | 5′-TTCCGTTTCTCGTGGTCACTT-3′ |
| Mouse COX-2 reverse | 5′-AGCGCTGAGGTTTTCCTGAA-3′ |
| Mouse haptoglobin | 5′-GCTGGGATCCTGAGCTTTGA-3′ |
| forward | |
| Mouse haptoglobin | 5′-TTGGCCATGGTTTCCTGAAC-3′ |
| reverse | |
| Mouse IL-1β forward | 5′-ACACTCCTTAGTCCTCGGCCA-3′ |
| Mouse IL-1β reverse | 5′-CCATCAGAGGCAAGGAGGAA-3′ |
| Mouse IL-2 forward | 5′-GAAACTCCCCAGGATGCTCAC-3′ |
| Mouse IL-2 reverse | 5′-GCCGCAGAGGTCCAAGTTC-3′ |
| Mouse IL-6 forward | 5′-AGAAGGAGTGGCTAAGGACCAA-3′ |
| Mouse IL-6 reverse | 5′-GGCATAACGCACTAGGTTTGC-3′ |
| Mouse IL-10 forward | 5′-GCCCTTTGCTATGGTGTCCTT-3′ |
| Mouse IL-10 reverse | 5′-TCCCTGGTTTCTCTTCCCAA-3′ |
| Mouse iNOS forward | 5′-TTCACCTCACTGTGGCCGT-3′ |
| Mouse iNOS reverse | 5′-GCACTCTCTTGCGGACCATC-3′ |
| Mouse MCP-1 forward | 5′-TCACAGTTGCCGGCTGG-3′ |
| Mouse MCP-1 reverse | 5′-TCTTTGGGACACCTGCTGCT-3′ |
| Mouse SAA-3 forward | 5′-CAGAAGTTCACGGGACATGGA-3′ |
| Mouse SAA-3 reverse | 5′-CCAGCAGGTCGGAAGTGGT-3′ |
| Mouse SOCS-1 forward | 5′-GCATCCCTCTTAACCCGGTACT-3′ |
| Mouse SOCS-1 reverse | 5′-AATAAGGGGCCCCCACTTA-3′ |
| Mouse SOCS-3 forward | 5′-AGGCACTCCCCGGGAGTAC-3′ |
| Mouse SOCS-3 reverse | 5′-GGCCACGTTGGAGGAGAGA-3′ |
| Mouse TNF-α forward | 5′-AAGGACTCAAATGGGCTTTCC-3′ |
| Mouse TNF-α reverse | 5′-CCTCATTCTGAGACAGAGGCAAC-3′ |
| Mouse cyclophilin A | 5′-TCGCCATTGCCAAGGAGTAG-3′ |
| forward | |
| Mouse cyclophilin A | 5′-GGTCACCCCATCAGATGGAA-3′ |
| reverse | |
| Mouse GAPDH forward | 5′-GGTTGTCTCCTGCGACTTCAA-3′ |
| Mouse GAPDH reverse | 5′-GTAGGCCATGAGGTCCACCA-3′ |
| Human COX-2 forward | 5′-TCCTGCCTACTGGAAGCCAA-3′ |
| Human COX-2 reverse | 5′-AGCCCTTCACGTTATTGCAGAT-3′ |
| Human IL-2 forward | 5′-GGGACTTAATCAGCAATATCAACGT- |
| | 3′ |
| Human IL-2 reverse | 5′-TTCTACAATGGTTGCTGTCTCATCT- |
| | 3′ |
| Human cyclophilin A | 5′-GCATACGGGTCCTGGCATC-3′ |
| forward | |
| Human cyclophilin A | 5′-TGCCATCCAACCACTCAGTCT-3′ |
| reverse | |
| Human GAPDH forward | 5′-ACCCACTCCTCCACCTTTGA-3′ |
| Human GAPDH reverse | 5′-CTGTTGCTGTAGCCAAATTCGT-3′ |

### Example 2

### Quantitation of Protein Using ELISA

The technique used to quantify the amount of a given protein in a liquid sample, either tissue culture supernatant or body fluid, is based on Enzyme Linked ImmunoSorbent Assay (ELISA) methodology. Either commercially available or established in house, the assay is based on the capture of the protein of interest by specific antibodies bound to the bottom of an ELISA plate well. Unbound material is washed away, the captured protein is then exposed to a secondary antibody generally labeled with horseradish peroxidase (HRP) or alkaline phosphatase (ALP). Again the unbound material is washed away, the samples are then incubated with the appropriate substrate yielding a colorimetric reaction. The reaction is stopped and reading is carried out in a spectrophotometer at the appropriate wavelength. Samples are tested at least in duplicate and the appropriate standard curve, consisting of serial dilutions of the recombinant target protein, is incorporated on each plate. Concentration of the protein in the sample is calculated from the standard curve.

### Example 3

### Quantitation of COX-2 Gene Expression in LPS Activated Macrophages and LPS Injected Mice Brains

We previously showed that dexanabinol and its analogs reduce the levels of secreted PGE₂ in LPS activated mouse macrophages (RAW 264.7) cells in vitro (WO 01/98289). The IC₅₀ for inhibition of PGE₂ secretion were determined and found to be 10 μM, 10 μM, 4 μM, and 8 μM, for dexanabinol, PRS-211,092, PRS-211,095 and PRS-211,220, respectively. For comparison, the IC₅₀ for inhibition of PGE₂ secretion for the known anti-inflammatory drugs Celecoxib, Rofecoxib and NS-398 were respectively 5 nM, 100 nM and 100 nM in the same experimental setup. We assumed then that this phenomenon observed at the level of PGE₂ secretion was due to direct inhibition of COX-2 enzymatic activity. To check this hypothesis we have now tested in vitro the enzymatic activity of recombinant COX-2 on its substrate in the presence of dexanabinol and its analogs PRS-211,092, PRS-211,095 and PRS-211,220. The assay reproduces in vitro the true enzymatic activity, the test compounds, up to 10 μM, were preincubated for 5 minutes with purified COX-2, then the natural substrate AA was added and the reaction was allowed to proceed for 2 minutes. HCl was used to stop the enzyme catalysis and saturated stannous chloride was added to stabilize the PGH₂ product into PGF_{2α}. The concentration of PGF_{2α} was measured using an Enzyme Immuno Assay (EIA), a standard curve was prepared and results were derived from this standard curve. Whereas classical COX-2 inhibitors such as VIOXX®, Indomethacin and NS-398 strongly inhibited COX-2 enzymatic activity in this assay, no inhibitory effect was observed for dexanabinol and its analogs. Therefore, we decided to test whether dexanabinol and its analogs have an unexpected impact on COX-2 gene expression and for this purpose we utilized LPS stimulated RAW 264.7 cells using real-time RT-PCR.

RAW 264.7 macrophages, a mouse cell line (ATCC # TIB 71), were grown in Dulbecco's modified Eagle's medium (DMEM) with 4 mM L-glutamine adjusted to contain 1.5 g/L sodium bicarbonate, 4.5 g/L glucose, and 10% heat inactivated fetal bovine serum. Cells were grown in tissue culture flasks and seeded at appropriate density into 24 wells tissue culture plates. 0.5×10⁶ Raw cells in one milliliter were stimulated with 2 μg/ml Lipopolysaccharide E. coli 055:B5 (DIFCO Laboratories). The mouse macrophages were pre-treated for one hour with controls or 10 μM of dexanabinol and its analogs, unless indicated otherwise, and later on activated with LPS. RNA samples were extracted from the cells 2.5 hrs after activation and COX-2 gene expression levels were analyzed by real-time RT-PCR as previously described.

The results of this experiment are expressed as fold activation of COX-2 over non-activated macrophages, after normalization to cyclophilin A expression. When cells are treated with vehicle only we observe a maximal 6-fold increase in COX-2 expression. A decrease of 67% in RNA levels of COX-2 is observed when the activation is carried out in presence of 10 μM dexanabinol. At this concentration all three analogs (PRS-211,092, PRS-211,095 and PRS-211,220) cause an inhibition of at least 50%. Together with the fact that COX-2 enzyme activity is not directly affected by these compounds, these results indicate that PGE₂ concentrations, that were originally monitored, were reduced as a result of decrease in COX-2 gene expression and not as previously thought by inhibition of enzymatic activity. However, at this stage we do not know if COX-2 gene regulation is directly achieved by our compounds, or results indirectly from the regulation of other genes with secondary impact on COX-2 through feedback mechanisms.

To test if the gene transcription regulatory effect of compounds of the invention was of physiological consequence in the whole animal, we injected either PBS or 100 ng LPS in vivo into mice brains intra cerebral ventricular (i.c.v.). Each treatment group was composed of at least five C57/BL male mice (6-8 weeks old, 25 g average body weight, Harlan, Israel). The mice were anesthetized with a mixture of 35 mg/kg pental and 8 mg/kg xylazine. LPS was dissolved in saline at 20 ng/μl and 5 μl were injected in each ventricule at a rate of 1 μl/min with the help of a syringe pump and a brain infusion canula. After each injection, the cannula is left in situ for one more minute to avoid reflux. The various treatment groups, controls including the cremophor:ethanol vehicle, dexanabinol and its analogs (20 mg/kg), were injected i.p. (0.1 ml/10 g body weight) simultaneously with the i.c.v injection of LPS. Six hours following LPS injection, the animals were sacrificed by i.p. injection of 100 mg/kg pentobarbitone sodium and their brains were removed and kept at −80° C. until next step. RNA was extracted from each whole brain and COX-2 gene expression levels were analyzed by real-time RT-PCR as previously described. The results of this experiment are expressed as fold activation of COX-2 in LPS versus PBS injected brains. LPS injected brains treated with vehicle only show a 5.3 fold increase in COX-2 RNA level, mice treated with 20 mg/kg dexanabinol display a reduction of 53% in the level of COX-2 activation.

From these experimental results, it is evident that the active ingredients of the present invention are effective in reducing COX-2 RNA level both in vitro and in vivo. Consequently, the compounds may be therapeutically effective in the wide variety of COX-2 related disorders.

### Example 4

### Quantitation of Secretion and Gene Expression in LPS Activated Macrophages

To test if COX-2 is the sole gene whose transcription is affected by compounds of the invention, additional genes were further analyzed by real-time RT-PCR in the above-described model of activated macrophages. This part of the study was performed with PRS-211,092 at doses up to 20 μM. The level of TNF-α and of MCP-1 was elevated by 19-fold and 55-fold respectively in activated as compared to resting macrophages, but PRS-211,092 did not affect the level of expression of any of these genes at the doses tested. The levels of iNOS and IL-1β were significantly elevated in activated macrophages by 266-fold and 18,960-fold respectively. PRS-211,092 inhibited gene expression in a dose dependent manner with an IC₅₀ of 4 μM for iNOS and of 17.5 μM for IL-1β. In this experiment COX-2 gene expression was elevated 70-fold by activation and under these conditions the dose related effect of PRS-211,092 yielded an IC₅₀ of 21.5 μM.

Moreover, it was confirmed that the reduction in IL-1β gene expression correlates to a reduction in IL-1β secretion. The IC₅₀ of dexanabinol, PRS-211,092 and PRS-211,220 for inhibition of IL-1β secretion are 6 μM, 3 μM and 4 μM, respectively. PRS-211,095 was tested at the single concentration of 10 μM and found to inhibit IL-1β secretion by 70%, similarly to PRS-211,092 and PRS-211,220 that both yielded 76% inhibition at that same concentration.

From these experimental results, it is evident that the active ingredients of the present invention are effective not only in affecting COX-2 RNA level but also in specifically reducing RNA levels of additional inflammatory related genes such as IL-1β and iNOS. As expected the impact on gene expression correlates with a decrease in secretion of the relevant inflammatory mediators. The fact that COX-2 is not the sole gene whose transcription is affected provide a significant advantage to compound of the invention over classical anti-inflammatory therapies which are designed to block one mediator at a time. A compound that can reduce the expression and secretion of multiple inflammatory mediators simultaneously harbors in a single molecule the approach of combined therapy now used to replace the single target classical anti-inflammatory strategies.

### Example 5

### Quantitation of IL-1β Gene Expression and Activated Microglia in LPS Injected Mice Brains

The local i.c.v. injection of LPS in mice brain stimulates a wide range of inflammatory responses and cascades where various cytokines and chemokines are implicated. Following the results obtained in examples 3 and 4, we decided to assess the effect of additional active compounds of the invention on other genes involved in the inflammatory response. Specifically, we tested the impact of dexanabinol and PRS-211,092, both at 20 mg/kg, on the expression of pro-inflammatory IL-1β cytokine. The protocol was basically as previously described with minor modifications. The amount of LPS injected in the mice brain was raised to 250 ng and the animals were sacrificed 24 hours after LPS and treatment injection for mRNA analysis, carried out as previously described. Moreover, some animals were kept alive and sacrificed 72 hours after LPS and treatment injection for immunohistochemical analysis. For this purpose, after euthanizing the animals by injecting i.p. 100 mg/kg of sodium pentobarbitone, brains were removed and fixed in 4% formaldehyde for at least 72 hours. Brains were then washed with PBS and transferred for cryoprotection in a solution of 30% sucrose in PBS, until they sank. After the brains sank in the sucrose they were frozen using the cryostat special fast-freezing technique (−60° C.). The brains were cryosectioned (18 μm) at the level of the entire hippocampus. Immunohistochemistry staining was carried out using polyclonal rat anti-mouse F4/80 (Serotec, USA) and goat anti-rat-IgG-peroxidase (Jackson, USA), for the detection of activated microglia cells. The slides were stained using the 3,3-diaminobenzidine tetrahydrochloride (DAB) chromagen detection kit of the automated immunostaining system (Vantana, France). Quantitative analysis was carried out by counting the number of immunoreactive cells/mm² at the level of the hippocampus.

Statistical Analysis

Results are expressed as mean±SD. Data were analyzed using analysis of variance (ANOVA) followed by post-hoc Fisher test and t-test. A value of p<0.05 is considered to be statistically significant.

Twenty-four hours following LPS injection, we observed a rise in the level of IL-1β mRNA, normalized to cyclophilin. LPS injected animals have 18 fold higher IL-1β mRNA levels than saline injected animals. Treatment with 20 mg/kg of PRS-211,092, administered simultaneously with the LPS stimulation, reduced the amount of IL-1β mRNA fold activation by 56% as compared to its vehicle.

Immunohistochemical analysis for the glial cell marker revealed that i.c.v. injection of LPS caused massive gliosis, restricted to the hippocampus. Sham operated animals displayed a baseline of zero activated microglia while LPS i.c.v. injected animals had on average as many as 488 immunoreactive cells/mm². Treatment with 20 mg/kg of PRS-211,092 significantly reduced the amount of activated microglia by 64%, while 20 mg/kg of dexanabinol caused a reduction of 40%, both as compared to animals treated with vehicle alone.

Altogether, these results show that active compounds of the invention act through modulation of inflammatory mediators in in vivo models of neuroinflammation as well as in in vitro models of activated cells of the immune system, such as the macrophages. Their effect is not only expressed by modulation of gene expression of soluble inflammatory mediators, but also by a significant decrease in the amount of activated cells involved in the inflammatory process.

### Example 6

### Quantitation of Gene Expression and Protein Secretion in Activated T Cells

IL-2 Gene Expression

The induction of the IL-2 gene is the hallmark event of T cell activation that is required for the resting cells to become effector cells. Similar mode of IL-2 gene regulation is obtained by T cell activation with PMA (phorbol-12-myristate-13-acetate) and calcium ionophore.

The human acute lymphoma T cell line Jurkat (ATCC # TIB 152) was used to test the possible immunosuppressive effect of dexanabinol and its analogs on T cell activation. The Jurkat cells were grown in RPMI 1640 medium with 2 mM L-glutamine adjusted to contain 1.5 g/L sodium bicarbonate, 4.5 g/L glucose, 10 mM HEPES, 1.0 mM sodium pyruvate, and 10% heat inactivated fetal bovine serum. Cells were grown in tissue culture flasks and seeded at appropriate density into 24 wells tissue culture plates. 2×10⁶ cells in one milliliter were stimulated using 10 ng/ml of PMA (Sigma) and 1 μM A23187 calcium ionophore (Sigma). Cyclosporin A (Sandoz), a known immunosuppressive drug, was used as positive control. The controls and test compounds were added at indicated concentrations one hour before stimulation. RNA samples were extracted from the cells 6 hrs after activation and IL-2 gene expression levels were analyzed by real-time RT-PCR as previously described.

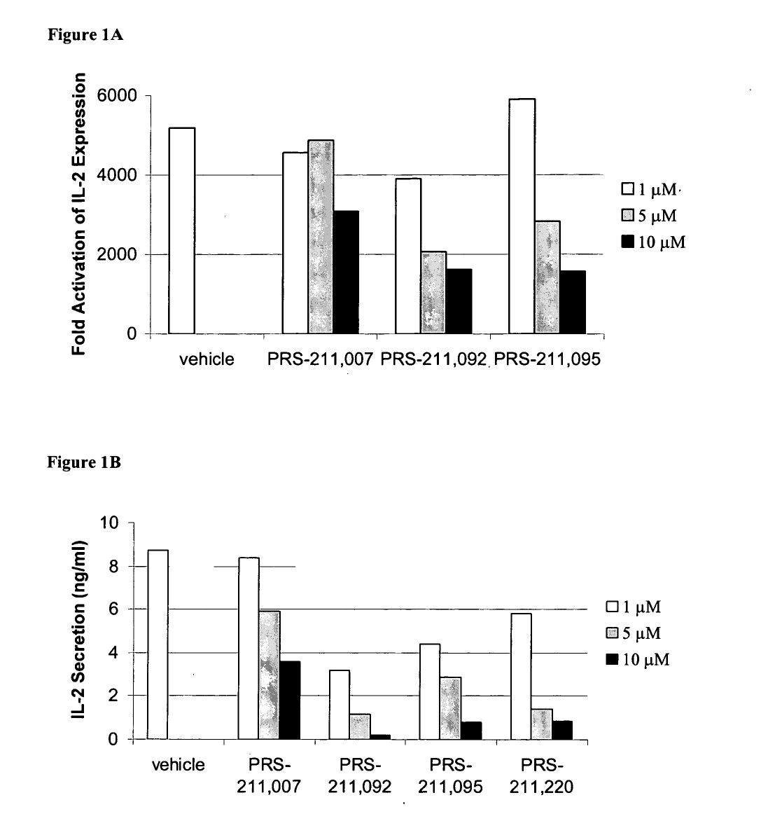

FIG. 1A depicts the result of this experiment as fold activation of IL-2 over non-activated T cells. The results are plotted after normalization to cyclophilin A expression. Dexanabinol and its analogs inhibited IL-2 gene expression in a dose related manner with maximal inhibitory activity at the highest dose tested, 10 μM. At this concentration dexanabinol, also known as PRS-211,007, reduced IL-2 fold of activation by 41%, PRS-211,092 by 69%, PRS-211,095 by 70% and PRS-211,220 by 84% (the latter not shown). Cyclosporin A completely blocked IL-2 transcription at 10 nM and inhibited it to 50% at 1 nM.

IL-2 Secretion

In parallel, the supernatant from each well was collected 24 hours after the activation and analyzed for the presence of secreted IL-2 by ELISA. The principles of the assay are as previously described. The secondary antibodies were conjugated with HRP, following substrate addition the peroxidase catalyzed color change is stopped by acidification. The absorbance measured at 450 nm is proportional to the concentration of IL-2 in the sample or standard. A standard curve is obtained by plotting the concentrations of recombinant IL-2 standards versus their absorbances. The IL-2 concentrations in experimental samples are then determined using the standard curve.

Results are shown in FIG. 1B, where the amount of secreted IL-2 (ng/ml) is plotted for each treatment group. When the activated cells are treated with vehicle only the maximal level of IL-2 secretion is 8.73 ng/ml. In correlation with gene expression inhibition, dexanabinol and its analogs PRS-211,092, PRS-211,095 and PRS-211,220 inhibited the secretion of IL-2 in a dose dependent manner at IC₅₀ of 8 μM, 0.4 μM, 1 μM and 2 μM respectively. For comparison, the positive control Cyclosporin A inhibited IL-2 concentrations in the growth medium with an IC₅₀ of 0.06 nM in the same experimental setup.

Taken together, these results indicate that dexanabinol and its analogs down-regulate IL-2, either directly or indirectly, both at the level of its RNA and at the level of its secretion, as was shown in activated Jurkat T cells. This implies that compounds of the invention may have a beneficial therapeutic impact on T cell mediated disorders.

COX-2 Gene Expression

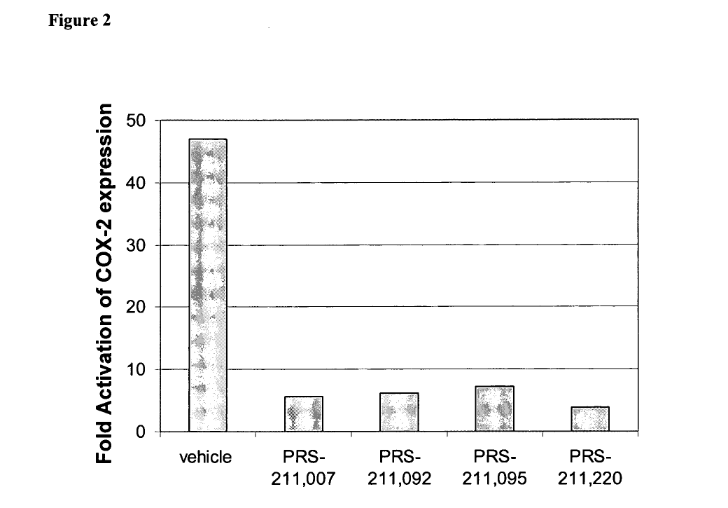

In addition, we used this experimental system to test the impact of dexanabinol and its analogs on COX-2 gene expression. Following activation of Jurkat cells with PMA and calcium ionophore we obtained elevated COX-2 gene expression levels. The impact of the test compounds on COX-2 was assessed as previously described for IL-2. FIG. 2 depicts the results of this experiment as fold activation of COX-2 over non-activated T cells. The results are plotted after normalization to GAPDH expression. We observe that COX-2 gene expression was inhibited by dexanabinol, also known as PRS-211,007, and PRS-211,092, PRS-211,095, and PRS-211,220, by 88%, 87%, 85% and 92% respectively. The positive control cyclosporin A was also very potent in this assay. COX-1 RNA levels were not changed throughout the experiment (data not shown) supporting the specificity of action of dexanabinol and its analogs.

From these experimental results, we confirm that the active ingredient of the present invention is effective in reducing COX-2 RNA level in another in vitro system, composed of T cells instead of macrophages. Moreover, these results prove that the effect of the compounds of the present invention is not limited to down-regulation at the level of gene expression, since the decrease in RNA is correlated to a decrease in protein secretion, as shown for IL-2. Consequently, the compounds may be therapeutically effective in the wide variety of COX-2 and IL-2 related disorders.

### Example 7

### Quantitation of Gene Expression and Protein Secretion in Activated Mast Cells

Mast cells are multifunctional bone marrow derived cells that upon activation release many potent inflammatory mediators. Release is done either from preformed granules, trough the process of degranulation, or following stimulation-induced de novo synthesis. The molecules released by Mast cells include biogenic amines such as histamine, chemokines, cytokines, enzymes, growth factors, peptides, arachidonic acid products and proteoglycans. It should be noted that mast cells are also known to play a key role in generating pain signal.

RBL-2H3 cells (ATCC # CRL-2256) are grown in EMEM medium with Earle's BSS, 2 mM L-glutamine adjusted to contain 1.5 g/L sodium bicarbonate, 0.1 mM non essential amino acids, 1.0 mM sodium pyruvate, and 15% heat inactivated fetal calf serum. Cells are grown in tissue culture flasks and seeded at appropriate density into 24 wells tissue culture plates. 2×10⁵ cells in one milliliter are seeded. Following overnight incubation, the plated cells are preincubated for half an hour with test compounds and controls and then stimulated with 10 ng/ml of PMA (Sigma) and 1 μM A23187 calcium ionophore (Sigma). The degranulation process is allowed to proceed at 37° C. for various periods of time, depending on the level of analysis and the molecule monitored. Cells are collected after half an hour for RNA preparation while supernatants are collected two and an half hours after stimulation, for the analysis of hexoaminidase and PGE₂ secretion, and IL-4 and TNF-α gene expression and secretion. For COX-2 gene expression RNA is collected one hour after stimulation. The Src family inhibitor PP2 or the PKC inhibitor GF109203X (both from Calbiochem) are used as positive control. The controls and test compounds are added at indicated concentrations before stimulation. The concentrations of the agent under study are measured in commercially available EIA, ELISA or enzymatic assays. Inhibition is calculated versus vehicle treated cells. In parallel, RNA samples are extracted from the cells at the appropriate predetermined time points after activation and gene expression levels are analyzed by real-time RT-PCR as previously described wherein the constitutively expressed COX-1 is used to normalize gene expression.

### Example 8

### Quantitation of Gene Expression in Brain Tissue Following Middle Cerebral Artery Occlusion

Transient MCAo in Mice

This model corresponds to cerebral ischemia as observed in stroke. Mice (C57/BL, male, 25 gr average body weight, Harlan, Israel) were anaesthetized with halothane in 30% oxygen and 70% nitrogen (4% for induction in an anesthesia chamber, and 1-2% in a facemask for maintenance). A midline incision was made in the skin of the neck, and the tissue underneath was bluntly dissected. The right common carotid artery (CCA) and its junction with the external carotid artery (ECA) and internal carotid artery (ICA) were explored by blunt dissection. The branches of the ECA, the occipital and the superior thyroid artery, were then cauterized. The CCA was then transiently closed by positioning around it a 5-0 silk suture material (Assut, Switzerland). Two cm pieces of the nylon suture material were cut and placed in a solution of 1% Poly-L-Lysine and then dried in an oven (60° C.) for 60 minutes. The tip of each piece was rounded under a flame. The ECA is permanently occluded with the same type of suture material. A third closure, transient this time, was done in the ICA with 5-0 silk suture material. A small hole is cut in the ECA and the nylon thread is inserted into the ICA while avoiding entrance into the pterygopalatine artery. The thread is inserted 11 mm until a slight resistance is felt. Then a 5-0 silk suture knot secures the thread. One cm of the thread left outside are then cut. The skin wound is closed by 5-0 silk suture material.

Following the operation, the animals were allowed to wake up in the cage. One-hour after insult initiation animals were clinically tested to verify the success of MCA occlusion. The evaluating system was based on works by Belayev et al., (Stroke 27: 1616-23, 1996; Brain Res. 833: 181-90, 1999). It consisted of two tests: the postural reflex test and the fore limb-placing test. The postural reflex was evaluated while the animal was suspended by the tail, whereas the fore limb-placing test was performed while the animal was held by the stomach. Table 1 summarizes the tests and their scoring system.

TABLE 1
Neurological evaluation of mice with MCAo.

| | Normal | |
|---|---|---|
| Item | Score | Deficit |
| Postural reflex test (hang test)* | 0 | 2 |
| Placing test (performed on each side)# | | |
| Visual placing | | |
| Forward | 0 | 2 |
| Sideways | 0 | 2 |
| Tactile placing | | |
| Dorsal surface of paw | 0 | 2 |
| Lateral surface of paw | 0 | 2 |
| Proprioceptive placing | 0 | 2 |

*Scores are as follows: 0 no observable deficit, 1 limb flexion during hang test, 2 deficit on lateral push.
#Scores are as follows: 0 complete immediate placing, 1 incomplete or delayed placing (>2 seconds), 2 absence of placing.

Only animals with total scores between 8 to 12 were included in the study. Ninety minutes after initiation of the insult, the selected animals are resedated using the same method, the neck wound is then re-opened and the nylon thread is pulled out of the ICA. The skin wound is then closed with 5-0 silk suture material. The controls and test compounds, (dexanabinol also known as PRS-211,007, PRS-211,092, PRS-211,095, and PRS-211,220) are administered 1 minute before the end of the insult. All treatments are delivered i.v. 5 mg/kg (except PRS-211,220 0.5 mg/kg). Vehicle is administered 5 ml/kg. Each treatment group comprised 6 to 8 animals. The drugs were dissolved in PEG-Ethanol and diluted in Intralipid (Pharmacia Upjohn). Eighteen hours later, animals were sacrificed by i.p. injection of pentobarbitone sodium 100 mg/kg. Brains were then removed, and total RNA was prepared from the ipsilateral half of the brains. Gene expression levels were analyzed by real-time RT-PCR as previously described. Results are expressed as fold activation over sham operated animals. Gene expression was normalized to house-keeping gene cyclophilin.

COX-2 Gene Expression in MCAo Brains

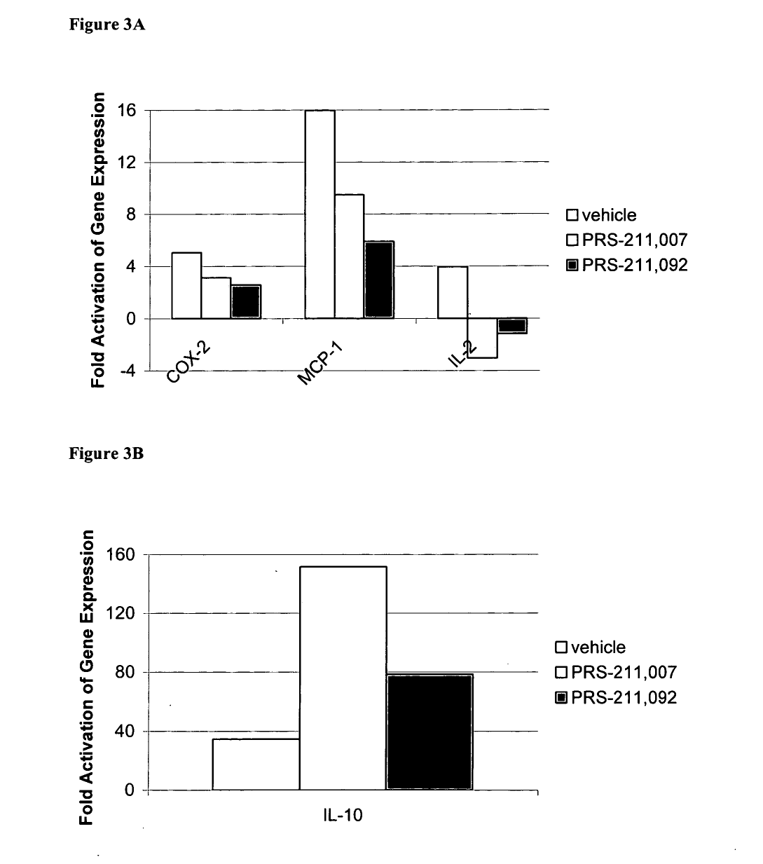

Dexanabinol and its analogs were already shown to be effective in reducing brain damage after stroke and improving outcome in the middle artery cerebral occlusion (MCAo) model in rats and mice. The purpose of this experiment was to check if these functional improvements were achieved by the newly identified mechanism of action. Therefore, COX-2 RNA levels were assessed 18 hrs following MCAo. The results obtained with all genes tested are displayed in FIG. 3. Vehicle only treated animals displayed a 5-fold activation of COX-2 gene expression versus sham operated animals. Treatment with dexanabinol and the PRS-211,092 clearly reduced this outcome by 38% and 48% respectively, in comparison with the vehicle treated group (FIG. 3A).

MCP-1 Gene Expression in MCAo Brains

Chemokines are low molecular weight, secreted proteins that chemoattractant and activate specific subpopulations of leukocytes. Monocyte chemoattractant protein-1 (MCP-1) is highly specific for monocytes, which are recruited to the site of injury, become activated and secrete inflammatory. Increased MCP-1 RNA levels following MCAo were previously reported (Che et al., Brain Research 902: 171-7, 2001). Immunohistochemistry studies showed that both ischemic neurons (after 12 hours of ischemic insult) and astrocytes (two days after insult) expressed MCP-1. We tested the effect of dexanabinol and PRS-211,092 treatments on MCP-1 RNA levels in mice brains after 18 hours of MCAo. Vehicle treated animals displayed a 16 fold increase in MCP-1 gene expression versus sham operated animals. Treatment with dexanabinol and PRS-211,092 reduced this outcome by 41% and 63% respectively, in comparison to the vehicle treated and the untreated groups (FIG. 3A).

IL-2 Gene Expression in MCAo Brains

Dexanabinol and its analogs were already shown to be effective in reducing IL-2 both at the level of gene expression and at the level of secretion in activated T cells. Therefore, we wished to verify their impact in vivo and we assessed IL-2 RNA levels in mice brains 18 hrs following MCAo. Vehicle only treated animals displayed a 4-fold activation of IL-2 gene expression versus sham operated animals. Treatment with dexanabinol and the PRS-211,092 clearly reduced this outcome by 177% and 130% respectively, in comparison with the vehicle treated group (FIG. 3A).

IL-10 Gene Expression in MCAo Brains

IL-10 is a potent anti-inflammatory cytokine strongly related to the previously described pro-inflammatory genes. Moreover, it has already been reported that IL-10 gene expression levels increase in rat brain following MCAo (Zhai et al, J. Neurol. Sci. 152: 119-24, 1997) and that IL-10 administration reduces rat brain injury following focal stroke (Sperat et al, Neurosci. Lett. 251: 189-92, 1998). We wish to check that this phenomenon can be repeated in the mice model of MCAo and that our compounds have a further positive impact on the expression of this anti-inflammatory cytokine. We tested the effect of dexanabinol and its analogs on IL-10 RNA levels in mice brains after 18 hours of MCAo. Vehicle treated animals displayed a 35 fold increase in IL-10 gene expression versus sham operated animals. Treatment with dexanabinol and PRS-211,092 further increased this outcome by 4.4-fold and 2.3-fold respectively, in comparison to the vehicle treated and the untreated groups (FIG. 3B).

Moreover, many more genes encoding for instance cytokines, adhesion molecules, or transcription factors, were reported to display abnormal levels of expression in cerebral ischemia. Out of a total of twenty four such agents screened in this study only the four genes previously described were affected by compounds of the invention. Surprisingly, in this model IL-1β, IL-6 and iNOS gene expression were not modulated by dexanabinol or PRS-211,092, at least not at the time point selected for analysis.

From these experimental results, we confirm that the active ingredient of the present invention is effective in reducing in vivo the RNA level of 3 important pro-inflammatory mediators, COX-2, IL-2 and MCP-1, while it is increasing the RNA level of the anti-inflammatory cytokine IL-10. Consequently, the compounds may be therapeutically effective in the wide variety of immune/inflammatory related disorders. Moreover, it should be noted that compounds of the invention are specific both in terms of the inflammatory related genes they regulate and in terms of the cellular targets or tissues in which they act.

### Example 9

### Quantitation of Gene Expression in Liver and Spleen Following ConA Injection in Mice

The ConA Model for T cell Mediated Injury

The most common causes of life threatening T cell mediated liver damage in humans are infections with hepatitis B or C viruses and autoimmune hepatitis. Different animal models of autoimmune liver injury have been developed, including acute liver failure in mice induced by intravenous injection of the T cell stimulatory plant lectin concanavalin A (ConA). ConA has high affinity for the hepatic sinus. Treatment of mice with ConA activates T cells that accumulate in the liver and release cytokines (IL-6, IL-10, TNF-α, INF-γ, IL-2) that regulate liver damage. Pretreatment with the immunosuppressor drugs such as cyclosporin A or FK506 completely prevents liver injury caused by ConA injection, demonstrating the major role of T cell activation in this model. We have shown that human T-lymphocytes (Jurkat cell line), activated by PMA and calcium ionophore, are inhibited by dexanabinol and its analogs (PRS-211,092, PRS-211,095 and PRS-211,220). Therefore, we decided to test in vivo the potency of these compounds to regulate T cell induced cytokine expression levels and reduce liver inflammatory damage using the ConA model.

Each experimental group contained at least twenty BALB/c inbred female mice (25 g average weight, Harlan, Israel). The negative control group was composed of mice injected with saline instead of ConA. The injection of ConA (Sigma) was done i.v. at the base of the tail at the dose of 15 mg/kg in saline. The treatments were injected i.v. at 5 mg/kg, 30 minutes before ConA injection, unless indicated otherwise. Compounds were dissolved in cremophor:ethanol, further diluted with saline before injection, and vehicle only was included as an internal control. Analysis was performed at predetermined time points following ConA injection.

Impact of treatment was monitored at three levels. First, blood samples (200-400 μl) were collected at predetermined time points after ConA injection, using retro-orbital puncture. After short centrifugation (5000 rpm for 2 min) serum was recovered and stored at −80° C. until further use for determination of cytokines concentrations by ELISA and aminotransferase release from the liver as a marker for liver injury.

In parallel, the concentration of cytokines was also determined in the organs of interests. For this purpose, the mice were killed by dislocation of the cervical vertebrae, at predetermined time points following ConA injection. The spleen and the liver were removed. Part of the liver was fixed in 4% formaldehyde for histology and the other part was kept at −80° C. for protein or RNA extraction. The spleens were weighted and a small part of the spleen was fixed in 4% formaldehyde, while most of the organ is cultured according to the following procedure. Each spleen is squeezed through a cell strainer with the rough end of a 5 ml syringe into 4 ml of RPMI medium. Large tissue fragments are removed by gravity sedimentation and the supernatants are collected. Cells are washed 3 times with 5 ml of erythrocyte lysis buffer (Boehringer), resuspended in 4 ml RPMI medium supplemented with 2 mM L-glutamine adjusted to contain 1.5 g/L sodium bicarbonate, 4.5 g/L glucose, 10 mM HEPES, 1.0 mM sodium pyruvate, and 10% heat inactivated fetal bovine serum, and plated in a 6 wells culture dish. Cells are incubated for 24 hours and cytokine concentrations in the supernatant were determined by ELISA as previously described.

ALT Release from ConA Injured Liver

Alanine aminotransferase (ALT) is an enzyme found mainly in the liver and it is measured to determine whether the liver is damaged or diseased in a variety of human conditions, especially hepatitis and cirrhosis. The level of its release into the bloodstream linearly correlates with the severity of the liver injury and thus it can be used both for diagnosis purposes and to monitor the efficiency of a treatment. The time course of plasma ALT release after ConA injection is known from the literature to peak at eight hours. At this time point the ALT concentration measured was about 1700 units/l and 5 mg/kg of PRS-211,092 when administered 30 minutes before ConA injection significantly reduced ALT concentrations by 57%. We used this assay to preliminarily assess the temporal window of PRS-211,092 efficiency and the compound was administered either 30 or 60 minutes after induction of injury. At both time points PRS-211,092 still reduced significantly ALT concentrations by 61% and 51% respectively. FK-506 was used as positive control and at the dose of 1 mg/kg, it reduced ALT concentrations by 92%, 93% and 86% when injected at the three previously described time points. Thus, the test compound has an important beneficial impact on liver injury as assessed by the reduction of ALT, a robust marker of the liver injury. This therapeutic effect is maintained even when the treatment is administered up to at least one hour after injury.

IL-2 Gene Expression in Liver and Spleen, and Secretion, in the ConA Model

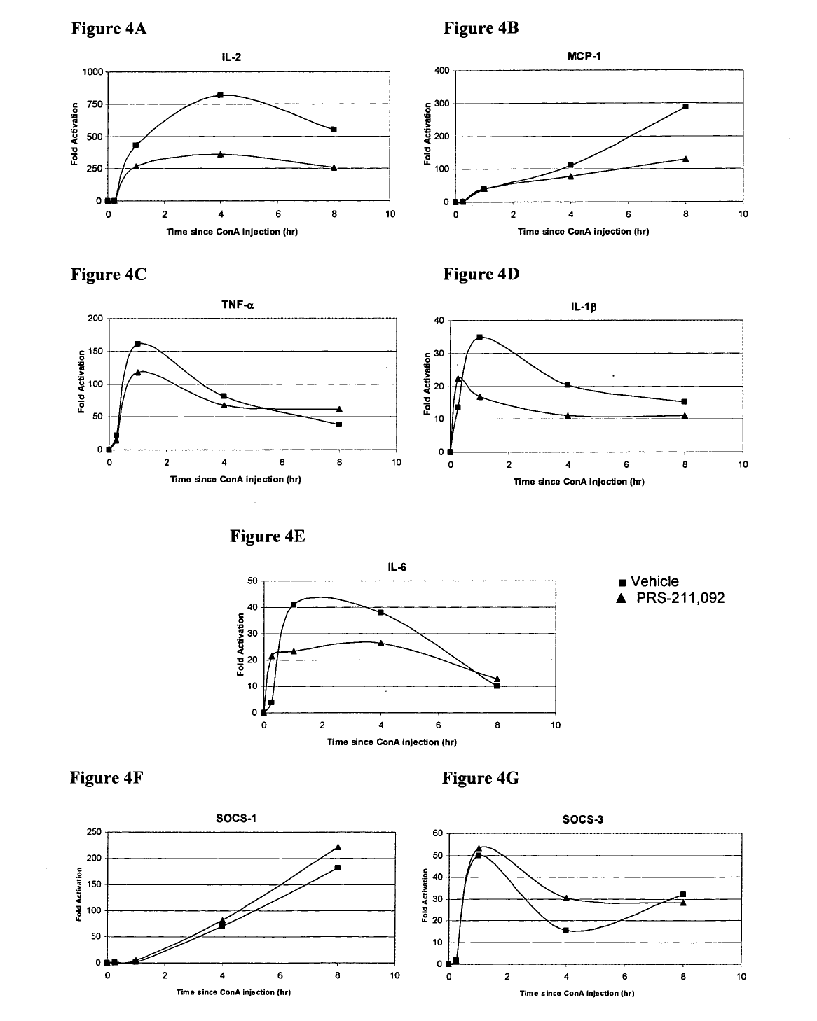

The induction of IL-2 expression in the liver of ConA injected mice and the effect of immunosuppressors in such a model have already been reported (Okamoto T. and Kobayashi T., Jpn. J. Pharmacol. 77: 261-3, 1998). The level of pro-inflammatory IL-2 expression was assessed in the liver at four time points: 15 minutes, 1, 4 and 8 hours following ConA injection. The effect of the compound is compared to its vehicle, which has no effect of its own on gene expression as assessed by the fact that vehicle treated animals yield results similar to saline treated animals (data not shown). Increase in IL-2 gene expression is detected only from one hour on (FIG. 4A). At this time point, vehicle treated animals displayed a 428-fold increase in IL-2 RNA levels versus saline injected animals. Treatment with 5 mg/kg i.v. of PRS-211,092 reduced this outcome by 37%. When the analysis is carried out 4 and 8 hours following ConA injection, the increase in IL-2 expression is correspondingly 817 and 549-fold and the test compound reduce these effects by 56% and 53% respectively. The effect of PRS-211,092 is statistically significant at all time points from one hour on.

Similarly, the level of IL-2 gene expression was assessed in the spleen of the ConA injected mice, one and four hours following injury. In this organ, vehicle treated animals displayed at each time point respectively a 309-fold and a 997-fold increase in IL-2 RNA levels versus saline injected animals. Treatment with 5 mg/kg i.v. of PRS-211,092 reduced this outcome by 46% when tested one hour after injury and by 32% when tested at four hours after injury.

In parallel, the spleen was removed and splenocytes were cultured for 24 hours to allow the assessment of secretion levels. Saline injected animals yielded undetectable background concentrations of IL-2, ConA injected animals treated with vehicle only yielded 1 ng/ml IL-2 while ConA injected animals treated with PRS-211,092 produced only 0.6 ng/ml, a 40% reduction in secretion from splenocytes. Moreover, the concentration of IL-2 was assessed in the plasma, PRS-211,092 succeeded to reduce the vehicle treated concentrations from 0.88 ng/ml down to 0.52 ng/ml, i.e. a 41% reduction. In this in vivo model we show that the reduction in IL-2 RNA levels is supported and correlated with a reduction in protein secretion. This was previously shown in the in vitro model of activated T cells.

MCP-1 Gene Expression in Liver in the ConA Model

Animals were sacrificed at the four time points following ConA injection previously described and the level of pro-inflammatory MCP-1 expression was assessed in the liver. Increase in MCP-1 gene expression is detected only from one hour on (40-fold increase over saline injected animals), but at this time point treatment with 5 mg/kg PRS-211,092 has no effect (FIG. 4B). Four and eight hours following ConA injection, the increase in MCP-1 expression is correspondingly 111 and 287-fold and the test compound reduces these effects by 29% and 55% respectively. The effect of PRS-211,092 is statistically significant at both time points.

TNF-α Gene Expression in Liver in the ConA Model

TNF-α, a cytokine produced mainly by activated macrophages, has pleiotropic effects both beneficial, as in liver regeneration, and deleterious, when it has direct cytotoxic role in human hepatocytes. TNF-α has been shown to be a crucial factor in immune mediated hepatitis and is also a mediator of hepatotoxicity in patients with alcoholic liver disease, fulminant hepatic failure and viral hepatitis. In most of these disorders, the concentration of TNF-α correlate inversely with patients survival.

Animals were sacrificed at the four time points previously described and the level of pro-inflammatory TNF-α expression was assessed in the liver. Increase in TNF-α gene expression is detected immediately, as early as 15 minutes following ConA injection (21-fold increase over saline injected animals), and already at this time point treatment with 5 mg/kg PRS-211,092 has an inhibitory effect of 32%. One, four and eight hours following ConA injection, the increase in TNF-α expression is correspondingly 162, 81 and 38-fold. The test compound reduces these effects by 27% and 17%, and then maintains the same level of TNF-α expression (FIG. 4C).

Moreover, it was confirmed in vitro that the reduction in TNF-α gene expression observed in vivo is indeed in correlation with a reduction in TNF-α secretion. For this purpose, the previously described experimental system of LPS activated macrophages was used. The IC₅₀ of dexanabinol, PRS-211,092, PRS-211,095 and PRS-211,220 for inhibition of TNF-α secretion are all between 10 to 20 μM.

IL-1β Gene Expression in Liver in the ConA Model

Animals were sacrificed at the four time points previously described and the level of pro-inflammatory IL-1β expression was assessed in the liver. Increase in IL-1β gene expression is detected immediately, as early as 15 minutes following ConA injection (14-fold increase over saline injected animals), but at this time point treatment with 5 mg/kg PRS-211,092 has an enhancing effect of 64% (FIG. 4D). One, four and eight hours following ConA injection, the increase in IL-1β expression is correspondingly 35, 20 and 15-fold. The test compound reduces these effects by 52%, 46% and 27%, respectively. The effect of PRS-211,092 is statistically significant at all time points. The effect of cytokine modulation might be in some cases a time dependent issue. At certain period, under given circumstances, the presence of a cytokine might be beneficial while it will become deleterious in another time window. The fact that IL-1β is first up-regulated may have an initial positive effect, such as induction of SOCS-1, further sustained by its later inhibition, as suggested by the decrease in ALT at the end of the study that supports the overall hepatoprotective activity of the test compound.

IL-6 Gene Expression in Liver and Spleen in the ConA Model

A bimodal role has been suggested for IL-6 in the ConA induced model of hepatitis. Some studies supported the claim that IL-6 production favors the development of hepatic injury, while in other studies IL-6 predominantly displayed a hepatoprotective activity. These contradictory observations have been reconciled when a thorough time course experiment was carried out with neutralizing antibodies. The results of this study indicate that IL-6 presence in the early phase of the disease is critical for evoking a strong hepatoprotective effect, while continued high concentrations of the cytokine are harmful for the liver (Tagawa Y-I et al., J. Leukoc. Biol. 67: 90-6, 2000).

Animals were sacrificed at the four time points previously described and the level of expression of the protective IL-6 cytokine was assessed in the liver. Increase in IL-6 gene expression is detected 15 minutes following ConA injection (4-fold increase over saline injected animals), and at this time point treatment with 5 mg/kg PRS-211,092 has an enhancing effect of 470% (FIG. 4E). One, four and eight hours following ConA injection, the increase in IL-6 expression is correspondingly 90, 38 and 10-fold. The test compound continues to have an enhancing effect on IL-6 gene expression but reduced to 38% at one hour after injury. At four hours PRS-211,092 reduces IL-6 ConA induced overexpression by 31%, and looses efficacy when the analysis is carried out eight hours after liver injury induction. The effect of PRS-211,092 is statistically significant at all time points. As observed in the case of IL-1β, PRS-211,092 has a bimodal effect on IL-6 expression. The fact that IL-6 is first up-regulated when it acts as a protective mediator and then inhibited when its hepatotoxic role predominates, supports the overall hepatoprotective activity of the test compound.

Preliminary studies carried out with the spleens of the ConA injected mice suggest that similarly to the situation observed in the liver, IL-6 gene expression is during the first hour after injury initially up-regulated by treatment with 5 mg/kg i.v. of PRS-211,092. Again, this up-regulation of IL-6 when the cytokine acts predominantly as a protective mediator is in line with the overall anti-inflammatory and liver protective activity of the test compound.

IL-10 Gene Expression in Liver and Spleen, and Secretion, in the ConA Model

The roles of the anti-inflammatory cytokine IL-10 in ConA model of murine liver injury have already been reported and it has been shown that administration of anti-IL-10 antibodies results in aggravated liver injury (Kato et al, Hepatology Research 20: 232-43, 2001). Animals were treated with 5 mg/kg i.v. of PRS-211,092 and euthanized 15 minutes, 1, 4 or 8 hours following ConA induced liver injury. Vehicle treated animals displayed on average a 9-fold increase in IL-10 liver or spleen RNA levels versus saline injected animals at all time points tested. Treated animals displayed further increased IL-10 gene expression by 1.5 fold in the spleen as early as 15 minutes after injury followed by a 3.2 fold increase 1 hr after injury. A similar increase of about 1.4 fold is observed in the liver only 8 hours after injury, suggesting that IL-10 is first up-regulated in the spleen then in the liver. In parallel to RNA quantitation in the whole organ, the spleen was removed at one hour after injury and splenocytes were cultured for 24 hours to allow the assessment of secretion levels. Saline injected animals yielded undetectable background levels of IL-10, ConA injected animals treated with vehicle only yielded 331 pg/ml IL-10 while ConA injected animals treated with PRS-211,092 produced 549 pg/ml, a 1.7 fold increase in secretion from splenocytes, which correlated with the increased RNA expression in the spleen. Last, the level of IL-10 was determined in plasma 1 hour following ConA injection and 5 mg/kg i.v. of PRS-211,092 significantly (p=0.03) increased the level of this anti-inflammatory cytokine from 493 pg/ml in vehicle treated animals to 726 pg/ml in PRS-211,092 treated animals, a 1.5 fold increase which also correlates with the increase in gene expression observed in the spleen at this early time point. In this in vivo model we show that the increase in IL-10 RNA levels is supported and correlated with an increase in protein secretion.

From these experimental results, we confirm in a second in vivo model that the active ingredient of the present invention is effective in reducing the RNA level of pro-inflammatory mediators while it is increasing the RNA level of the anti-inflammatory cytokine IL-10. Consequently, it further supports the fact that compounds of the invention may be therapeutically effective in the wide variety of immune/inflammatory related disorders.

Expression of Genes Involved in Cytokine Signal Transduction, in the ConA Model

The purpose of this study was to determine if a common mechanism lye beyond the observations that active compounds of the invention are efficient modulators of anti- and pro-inflammatory mediators. In recent years, progress was made in unraveling cytokine signal transduction. This work led to the recognition of Janus kinases (JAKs) and signal transducers and activators of transcription (STATs) as positive regulators; and of suppressors of cytokine signaling (SOCS), protein inhibitors of activated STATs (PIAS) and the SH-2 containing phosphatase, as negative regulators of this signaling pathway. In this study we checked the effect of ConA induced liver injury on the levels of SOCS-1 and SOCS-3, as previously described. Both SOCS-1 and SOCS-3 have important recognized roles in the liver. SOCS-1 deficient mice suffer from three major abnormalities: lymphopoenia, macrophage infiltration of several organs including the liver, heart, lung and skin, and severe fatty degeneration of the liver. On the other hand, SOCS-3 is the main SOCS gene induced in the liver by GH. Thus, we tested if PRS-211,092 had an effect on the expression of these negative regulators of gene expression.

As can be seen from FIGS. 4F and 4G, the pattern of impact on gene expression is a mirror image of the impact of PRS-211,092 on the cytokine themselves. While PRS-211,092 lowered the almost linear slope of MCP-1 expression over time from liver injury, it symmetrically increased the slope of SOCS-1 gene expression. Similarly the pattern of increase in SOCS-3 due to PRS-211,092 treatment is the mirror image of the decrease in IL-2, TNF-α, IL-1β, and IL-6. These results, showing activity of the test compound on the modulation of regulators of cytokine signal transduction, support the fact that the active compounds of the invention act through regulation of transcription of genes involved in inflammatory processes.

Moreover, when taken together with the results obtained with the downstream regulated cytokines and chemokines, we can make the following observation. From the graphs displayed in FIG. 4 we see that generally speaking PRS-211,092 has a clear moderating effect on the expression of inflammatory related genes, or their regulators, that are activated during the course of liver injury in the ConA model. PRS-211,092 down-regulates the expression of positive inflammatory mediators and up-regulates the expression of negative mediators. The cytokines and chemokine tested often act in concert and cross regulate one another to yield the final physiological outcome. When the ultimate effect of the above-described gene modulation is tested by a biological marker of liver injury, such as ALT, SAA-3 and haptoglobin, we conclude that the overall end result is indeed hepatoprotection.

Expression of Genes Involved in Acute Phase Response, in the ConA Model

The acute phase response is an innate body defense seen during acute inflammation, infection and trauma, which involves the altered production of certain blood proteins termed acute phase proteins (APPs). As previously stated activated macrophages and other leukocytes release pro-inflammatory cytokines such as TNF-α, IL-1 and IL-6. These cytokines in turn stimulate hepatocytes to synthesize and secrete acute phase proteins such as C-reactive protein (CRP), mannose-binding lectin (MBL), haptoglobin and serum amyloid A (SAA). Measurement of acute phase protein level is used to monitor the severity of the innate response and thus predict the prognosis of the disease. The purpose of this study was to determine if indeed the modulation of anti- and pro-inflammatory mediators in the liver lead to the appropriate down-regulation of positive APPs, both at the level of gene expression and secretion. In this study we checked the effect of ConA induced liver injury on the levels of SAA-3 and haptoglobin, and we tested if PRS-211,092 had an effect on the expression of these acute phase proteins, as previously described.

Administration of 5 mg/kg PRS-211,092, 1 mg/kg FK-506 or vehicle thirty minutes before ConA had the following effects on the APPs tested four and eight hours after induction of liver injury. PRS-211,092 significantly reduced the levels of liver gene expression of SAA-3 by 55% and 48%, and of haptoglobin by 56% and 66%, at the respective time points. The positive control FK-506 yielded on average at both time points of four and eight hours after injury 97% and 78% reduction in gene expression of SAA-3 and haptoglobin respectively. These results further strengthen the fact that the modulatory effect of active compound of the invention on gene expression of the cytokine network of pro- and anti-inflammatory mediators has an overall therapeutic benefit, as monitored by the significant decrease in the APPs, SAA-3 and haptoglobin.

### Example 10

### Hepatoprotection Through Inhibition of Apoptosis in HepG2 Cell Lines

The purpose of this study is to assess whether the hepatoprotective effect of the active compounds of the invention is achieved through inhibition of apoptotic events in cells of hepatic lineage. The proteolytic cleavage of caspases and poly-ADP-ribose polymerase (PARP) is a known marker of apoptosis, and is investigated by immunoblotting or immunohistochemistry in HepG2 cells. Apoptosis is induced in these cells either by TNF-α, anti-CD95 or ethanol.

HepG2 (ATCC # HB-8065) are human hepatocellular carcinoma cells and are grown in Eagle's Minimum Essential medium, supplemented with 10% FCS, 2 mM L-glutamine, 1 mM sodium pyruvate, 0.1 mM nonessential amino acids, and 1.5 g/l sodium bicarbonate. A total of 1×10⁶ cells/well are seeded in 6-well plates and are treated with either anti-CD95 (0.5 mg/ml) or with TNF-α (10 ng/ml), and cycloheximide (10 mg/ml). For ethanol induced apoptosis, the range of the inducer is 100 to 400 μM and cells must be cultured for 24 hours under those conditions. Test compounds and controls are added to the cells one hour before the induction of apoptosis, unless otherwise stated. After 6 hours for TNF-α or anti-CD95 or 24 hours for ethanol induction, cells are washed in cold PBS and lysed in 1% Triton X-100, 50 mM Tris-HCl, pH 7.6, and 150 mM NaCl containing 3 mg/ml leupeptin, 3 mg/ml aprotinin, 3 mg/ml pepstatin A and 2 mM phenylmethylsulfonyl .uoride. After centrifugation (10 minutes, 13,000 rpm, 4° C.), the cell lysates are separated by sodium dodecyl sulfate polyacrylamide gel electrophoresis and transferred onto a polyvinylidene di.uoride membrane (Amersham, Germany). Membranes are blocked with 5% milk powder in Tris-buffered saline and then incubated for 1 hour with 1 mg/ml of either anti-cleaved caspase-3, anti-cleaved caspase-7, or antibodies recognizing the full-length forms of the proteins. Membranes are then washed 4 times with Tris-buffered saline/0.05% Tween-20 and incubated with the respective peroxidase-conjugated secondary antibodies for 1 hour. After extensive washing, bound antibodies are detected by enhanced chemiluminescent staining.

### Example 11

### Inhibition of IL-2 Related Transcription Factors in Activated T Cells

We have established that IL-2 gene expression and secretion are inhibited by the active compounds of the invention both in vitro, in PMA/Calcium ionophore activated T cells, and in vivo in the MCAo model for determination of neuroprotection and in the ConA induced model of liver injury for determination of hepatoprotection. IL-2 is tightly regulated at the level of transcription. The purpose of this study was to check if the test compounds have an effect on some of the transcription factors involved in the regulation of the IL-2 promoter.

Two different lines were established, in each case Jurkat cells were cotransfected with a plasmid containing a luciferase reporter gene controlled by either NF-AT (Clontech cat# S2088) or AP-1 (Clontech cat# S2087) transcription elements and with a plasmid containing the neomycin resistance gene. Transfection was carried out using the cationic lipid reagent method (Dimeri-C, Invitrogen Life Technologies). In order to isolate stable clones, the transfected human T cells were submitted to gentamicin selection for four weeks, and positive clones were identified. The stable clones, transfected with the various transcription elements controlling luciferase, were grown as previously described for Jurkat cells, with the addition of 300 μg/ml gentamicin. On the experimental day, the cells containing each reporter were plated in a 24 well plate at 10⁶ cells/ml/well. Test compounds were resuspended in DMSO, and added for one hour before cell activation, at predetermined doses wherein DMSO final concentration is 0.1%. Cyclosporin A was used as positive control at the dose of 100 nM. Cells were then stimulated with 10 ng/ml PMA and 2 μM calcium ionophore for 6 hours. At the end of the experiment, cells were collected, rinsed in PBS and lyzed for 15 minutes on ice in 50 μl luciferase lysis buffer (Promega). Cell debris were removed by spinning down the cell lysate for 5 minutes at high speed at 4° C. Luciferase activity was measured in a black 96 well plate, where 10 μl of cleared cell lysate were combined with 90 μL of luciferin substrate (Promega). Luminescence was immediately measured in the appropriate reader (X-flour, Tecan).

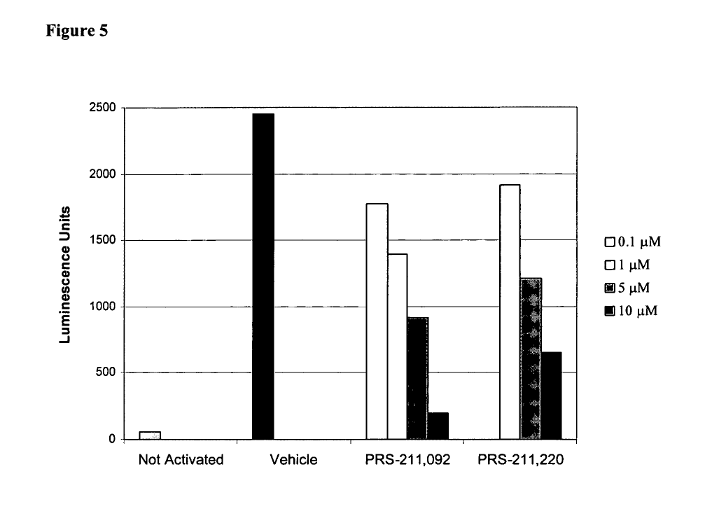

Results are shown in FIG. 5, where we can see that activation of the cells yield a significant increase in NF-AT driven luciferase expression, from 60 luminescence units (LU) to 2449 LU. Treatment of the cells with test compounds caused a dose dependent reduction in NF-AT driven luciferase expression. PRS-211,092 has an IC₅₀ of 1.5 μM and PRS-211,220 has an IC₅₀ of 3.9 μM. In this experimental setup, 100 nM of cyclosporin A caused a total inhibition. Similarly, we tested the activity of 10 μM PRS-211,092 on cells stably transfected with the AP-1 reporter. Cell activation increased the level of AP-1 driven luciferase expression from 182 LU to 19391 LU, however neither PRS-211,092 nor cyclosporin A affected this outcome. These results further support that the active compounds of the invention act through regulation of transcription of genes involved in inflammatory processes.

### Example 12

### DNA Arrays

DNA-array based technologies are widely used in gene regulation research, most commonly to measure differential gene expression, that is comparing the relative level of RNA transcripts in different samples. The purpose of this study is to allow a preliminary screen of the impact of dexanabinol and its analogs on the regulation of a large amount of genes. The technology is based on hundreds (macro-arrays) to thousands (micro-arrays) of sequence-specific DNA fragments spotted on a solid matrix such as glass slides or membranes. RNA samples from the examined tissue or cells are reverse-transcribed into cDNA, labeled and hybridized with the array. The number of labeled transcripts hybridized to a single spot is turned to a radioactive, fluorescence or chemiluminescence signal and detected by the appropriate instrument. The quantification of the signal on each spot measures the level of expression of the specific gene. We have used membrane-based focused macroarrays each consisting of gene families representing a biological regulatory pathway such as cytokines or chemokine arrays, commercially made by SuperArray Inc. cDNA samples from mice brains after MCAo treated with either vehicle or test compound were labeled with biotin, hybridized to the array-membranes and detected using a chemiluminescence detector, according to SuperArray instructions. The genes that are expressed differentially between treatment and control are subjected to confirmation analysis using real-time quantitative PCR, as previously described.

B-Impact of Tricyclic Dextrocannabinoids on Animal Models for Various Diseases

### Example 13

### Effect of the Compounds in Carrageenan Induced Paw Edema

The purpose of this study is to test in vivo the anti-inflammatory activity of the compounds in paw edema induced by injection of 1% carrageenan in the animal hind paw. Female Balb/c mice (20 gr average body weight, Harlan, Israel) are anesthetized with a combination of xylazine and pentobarbitone diluted in sterile saline, 15 and 6 mg/kg i.p. respectively. Anesthetized mice are injected subcutaneously, in the subplantar region of one (right) paw with 0.05 ml of 1% w/v Carrageenan in sterile water. The contralateral (left) paw is not injected as data from the literature, confirmed by our own experience, showed that injection of 0.05 ml of normal saline did not affect later thickness or volume measurements. The test compounds, including known anti-inflammatory controls, are dissolved in cremophor:ethanol and further diluted 1:20 or 1:50 in sterile saline before i.p. injection that takes place immediately before the carrageenan injection. Three hours after injection the animals are resedated following the previously described procedure. Paw thickness is measured using a dial thickness gauge (Spring-dial, constant low pressure gauge, Mitutoyo, TG/L-1, 0.01 mm) and paw volume is measured using a plethysmometer (model #7150, Ugo Basile, Italy). Paw Edema is expressed as the difference between the right treated and the left untreated paws of the same animal, either as Δ Paw Volume (ΔPV) in millimeters cube or as Δ Paw Thickness (ΔPT) in millimeters. Each group comprises at least 10 animals. Results can be further normalized to the ΔPV and ΔPT values of each treatment group at 0 mg/kg (vehicle only). At the end of the study, animals are euthanized with an i.p. injection of 100 mg/kg pentobarbitone.

The differences between ΔPV and ΔPT among various treatment groups are analyzed by analysis of variance (ANOVA) followed by post-hoc Fisher test. A value of p<0.05 is considered to be statistically significant.

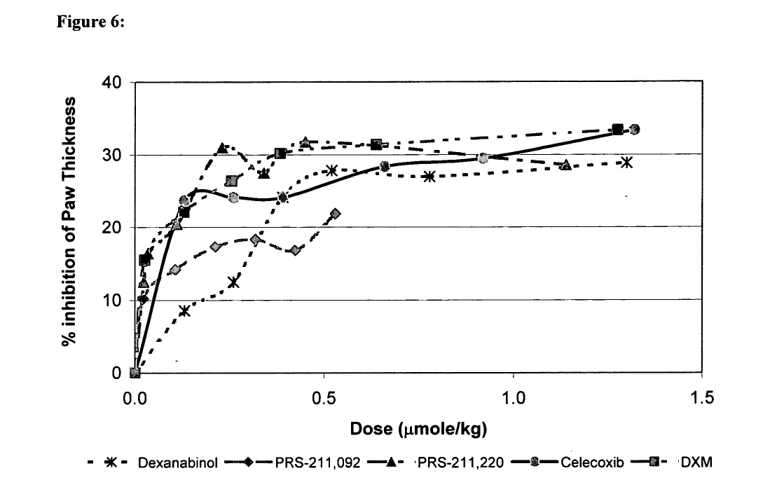

Results are depicted in FIG. 6 where the % inhibition of paw thickness, normalized to vehicle, is plotted against the dose of the test compound. Dexanabinol yields a reduction of about 29% in paw thickness at doses ranging from 0.2 to 0.5 mg/kg. PRS-211,092 yields a reduction of about 22% in paw thickness at the very low dose of 0.25 mg/kg. PRS-211,220 reduces even more paw thickness by 31% at the even lower dose of 0.1 mg/kg. These results are statistically significant as compared to vehicle treated animals. At these doses, known anti-inflammatory drugs such as Celecoxib and Dexamethasone (DXM) yield respectively 24% and 26% reduction in paw thickness at 0.1 mg/kg, 28% and 31% at 0.25 mg/kg and both 33% at 0.5 mg/kg. It should be kept in mind that these commercially available drugs display serious side effects that prevent chronic uses without complementary protective medication. The fact that compounds of the invention have anti-inflammatory activity comparable to these drugs is very encouraging since compounds of this family have the advantage of being devoid of side effects, thus making them interesting candidates for the replacement of existing anti-inflammatory drugs. These results support that the active compounds of this invention, which act through modulation of pro/anti-inflammatory mediators, have an anti-inflammatory effect that may be relevant to a wide range of human conditions with inflammatory components.

### Example 14

### Effect of Compounds in Cancer Chemoprotection

In vitro.

Cells from several tumor-derived cell lines are tested for their proliferation capacity in presence of our test compounds. Pancreatic tumor cell lines were obtained from ATCC. Panc-1 (ATCC # CRL-1469) were cultured in DMEM supplemented with 4 mM Glutamine, 4.5 g/L glucose, 1.5 g/L bicarbonate, antibiotics (penicillin, nystatin and streptomycin) and 10% heat inactivated fetal calf serum. Aspc-1 (ATCC # CRL-1682) were cultured in RPMI supplemented with 2 mM Glutamine, 10 mM HEPES, 1 mM sodium pyruvate, 4.5 g/L glucose, 1.5 g/L bicarbonate, antibiotics (penicillin, nystatin and streptomycin) and 20% heat inactivated fetal calf serum. Cells were seeded in a 24 well plate (10⁵ cells/ml/well) and grown overnight. The cells are incubated with the test compounds (1-100 μM) or vehicle (0.1% DMSO final concentration). Cell viability was determined 24 hours later using standard crystal violet staining. The culture medium was removed from the wells and the cells were fixed by adding 1 ml/well of 2% formaldehyde in PBS for 10 minutes. Following fixation the cells are washed three times with PBS and 250 μl of 0.5% (w/v) crystal violet is added to each well and the plates were incubated for 30 minutes at room temperature with gentle agitation. The stained cells were then washed three times with double distilled water and the color was extracted by adding to each well 250 μl of 10% acetic acid. The plates were agitated for 15 minutes at room temperature and 100 μl were transferred in duplicate to a 96 well plate for reading. Optical density (OD) of the cells was measured at 620 nm in an ELISA reader and results are expressed as % viable cells. Absorbance of untreated cells is recorded as 100%. The experimental results show that Aspc-1 proliferation was not affected by the presence of dexanabinol up to 15 μM whereas Panc-1 cells proliferation was inhibited by 26% at this same concentration. The IC₅₀ (dose inhibiting cell growth by 50%) is determined.

Moreover, the cells are stained for activated caspase 3 to determine whether they died through an apoptotic mechanism. The medium from the wells is discarded and cells are fixed by adding 1 ml of 4% formaldehyde in PBS, for 10 min. Cells are washed twice with PBS-0.1% Tween20 (PBS-T) and permeabilized with cold methanol for 20 min. The cells are washed twice with PBS-T and incubated with 1 ml blocking solution (3% BSA, PBS-T) for 30 min. The primary antibody (rabbit anti- cleaved caspase 3 (asp175) Cell Signaling Technology, diluted 1:50 with blocking solution) is added and the- cells incubated for 60 min. at 37° C. The cells are washed twice with PBS-T. The secondary antibody (HRP conjugated anti-rabbit IgG diluted 1:200 with blocking solution) is added to the wells and incubated for 60 min. at RT. Cells are washed twice with PBS-T and incubated for 10 min with a fluoresceine tyramide reagent (NEN, diluted 1:50 with amplification diluent). Cells are washed twice with PBS-T and the signal visualized by fluorescence or confocal microscope. Beside monitoring activated caspase-3, the expression of apoptosis-related genes in cells treated with dexanabinol and its analogs is compared to that in untreated cells. The procedure for real-time RT-PCR is as previously described. For each gene, a pair of specific PCR primers is designed and the reaction is done according to the ABI protocols. The quantification of each gene expression level is normalized to a housekeeping gene and compared to RNA samples from non-treated cells.

In vivo.

Once we have shown that dexanabinol and its analogs were inhibitors of tumor cell proliferation in vitro, we wished to test if our compounds were as efficient in vivo. For this purpose the tumor cells LoVo (originating from a colorectal tumor; ATCC # CCL-229) were grown in DMEM containing 4.5 g/L glucose, 2 mM Glutamine, 1% Pen/Strep and 10% heat inactivated fetal calf serum. The cells were harvested using Trypsin-EDTA, the detached cells were rinsed in PBS, and counted. Predetermined amounts (1×10⁶ cells in constant volume of 0.12 m/animal) were injected s.c. above the right femoral joint in nude CD-1 male mice (average weight 20-25 gr, Harlan, Israel). Each treatment group was composed of at least 7 animals. Each animal was clinically monitored daily. The growth of the tumor was also monitored during the daily visits but actual measurements were recorded once a week. When tumors reach the appropriate size, animals are treated with either vehicle, 5 ml/kg/day, or with our test compounds, in the range of 2.5 to 10 mg/kg/day.

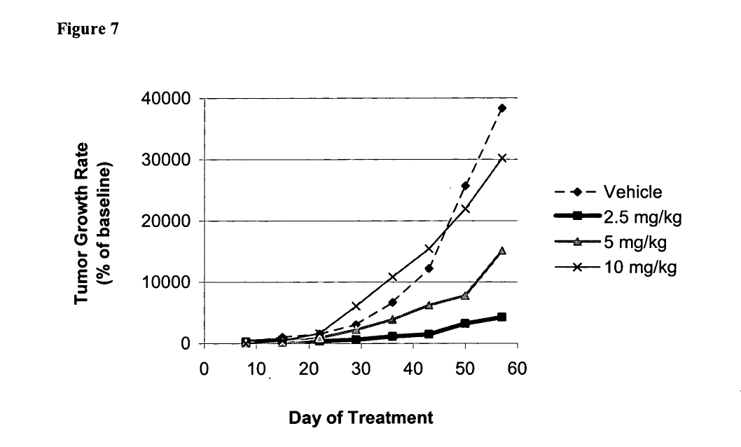

Thirty-six out of the 40 implanted mice developed a visible tumor within 5 to 6 weeks from tumor implantation. All animals were treated first on the 36^{th}-42^{nd} day of tumor implantation and the treatment lasted 8 weeks. One animal died in each treatment group between the 5^{th} and the 7^{th} week of treatment. The results are expressed as percent of tumor growth at the various days of the treatment as compared to baseline day 1. The results are depicted in FIG. 7 where we can observe that the efficacy of dexanabinol is inversely proportional to its dose in the range tested. 2.5 mg/kg (▪) seems more efficient than 5 mg/kg (▴), itself better than 10 mg/kg (x), which has very little effect, if at all, as compared to vehicle (--♦--). By the end of the treatment, 2.5 mg/kg dexanabinol reduced the tumor growth by 89%, 5 mg/kg by 61% and 10 mg/kg by 21%. These experimental results strongly suggest that the active compound of this invention, which acts through modulation of pro/anti-inflammatory mediators, may be therapeutically effective against certain types of tumors.

### Example 15

### Treatment of Neurodegenerative Disorders: the MPTP Model

Parkinson's disease (PD) is a neurodegenerative disorder characterized by tremor, slowness of movements, stiffness and poor balance. Most, if not all, of these disabilities are due to a profound reduction in striatal dopamine content caused by loss of dopaminergic neurons in the Substantia Nigra pars compacta (SNpc) and of their projecting nerve fibers in the striatum. 1-Methyl-4-phenyl-1,2,3,6-tetrahydropyridine (MPTP) is a well known neurotoxin that can cause depletion of dopamine content in the striatum and a reduction in the number of nigrostriatal dopaminergic neurons in several species including humans (Turski L. et al, Nature 349: 573, 1991). Neuroinflammatory phenomena are observed in this model. The aim of the present study is to examine the effect of compounds involved in gene regulation of pro- and anti-inflammatory mediators on the progression of MPTP-induced dopaminergic toxicity. The study is carried out in two time windows. In the short-term model, the neurological outcome is assessed, namely by measuring the effect of the test compounds on the number of immunoreactive cells. In the long-term model, the functional outcome is assessed in the rotarod system following various treatments.

Animal Treatment and Procedure: the Short-term Model for Neurological Outcome

The mice (C57/BL male mice, average weight 30 g, Harlan, Israel) were administered i.p. with 4 injections of MPTP (Sigma, USA) (20 mg/kg, 5 ml/kg) in saline (Teva Medical Israel) at 2 hours interval on day 1. The test compounds and vehicle control (cremophor:ethanol diluted in saline) were injected i.p. once just before the first MPTP administration at a volume dose of 5 ml/kg. Dexanabinol was tested at 10, 20, and 30 mg/kg and PRS-211,220 was tested at 0.5, 1, and 5 mg/kg. Each treatment group was composed of at least seven animals. Seven days following the MPTP treatment the animals are euthanized by i.p. administration of 100 mg/kg pentobarbitone sodium (CTS, Israel) and their brains are removed for tyrosine hydroxylase (TH) detection using immunohistochemistry.

TH Immunoreactivity at the Level of the SNpc

Brains were fixed by immersion in 4% formaldehyde for at least 72 hours. The brains were then washed with PBS and transferred to 30% sucrose in PBS until they sank. After the brains sank in the sucrose they were frozen using the cryostat special fast-freezing technique (−60° C.). The brains were cryosectioned (20 μm) at the level of the striatum and at the level of the substantia nigra (SN). Immunohistochemistry staining was carried out using Rabbit anti-tyrosine hydroxylase (1:250, Calbiochem, USA). The slides were stained using the 3,3-diaminobenzidine tetrahydrochloride (DAB) chromagen detection kit of the automated immunostaining system (Vantana, France). Quantitative analysis was carried out by counting the number of TH-immunoreactive cells/mm² at the widest area of the SNpc.

Statistical Analysis

Results are expressed as mean±D. Data were analyzed using analysis of variance (ANOVA) followed by post-hoc Fisher test. A value of p<0.05 is considered to be statistically significant.

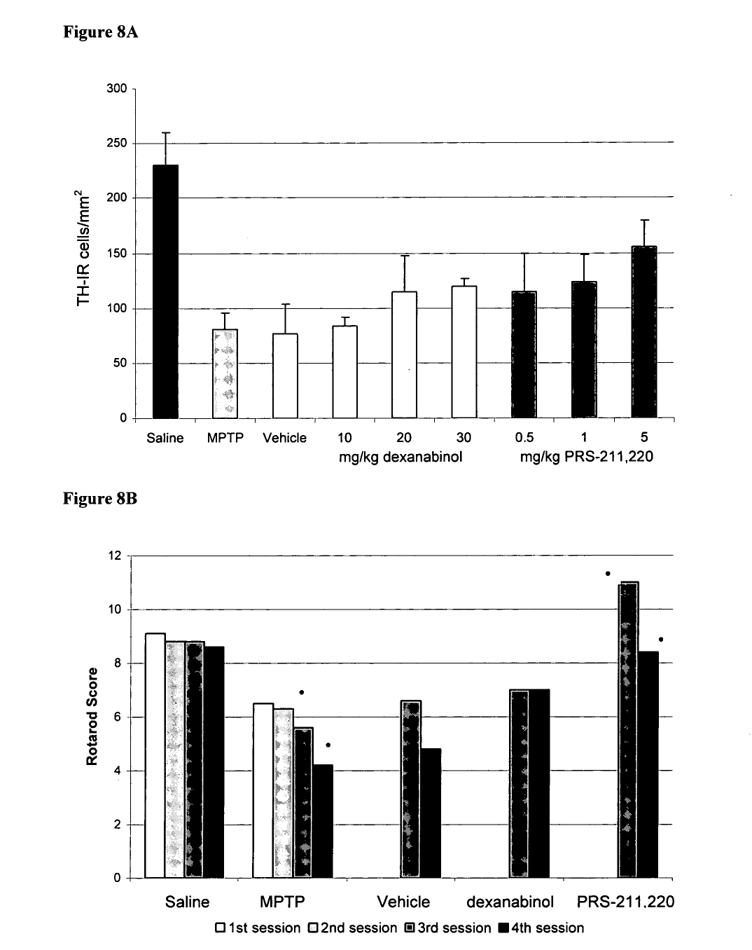

The results are depicted in FIG. 8A, where we can see that MPTP injections resulted in a reduction of about 65% in the number of TH-IR cells at the SNpc level as compared to the saline injected animals. Treatment of the animals with the test compounds results in a dose-dependent preservation of the TH-IR cells. Percent preservation is calculated by dividing the number of rescued cells (treatment-MPTP) by the maximal possible number of rescued cells (saline-MPTP). Best rescue were observed with 20 mg/kg of dexanabinol, which preserved about 25% of the TH-IR cells, increasing the dose to 30 mg/kg having no significant effect, and 5 mg/kg of PRS-211,220, which preserved approximately 50% of the TH-IR cells. Vehicle alone had no effect on TH-IR preservation. These two doses of test compounds were further used to determine the functional outcome.

Animal Treatment and Procedure: the Long-term Model for Functional Outcome

The mice (C57/BL male mice, average weight 30 g, Harlan, Israel) were administered i.p. with MPTP (Sigma, USA) (40 mg/kg, 5 ml/kg) in saline (Teva Medical, Israel) at 2 weeks interval for four times. The test compounds and controls were injected i.p. once just before the first MPTP administration at a volume dose of 5 ml/kg. Dexanabinol was tested at 20 mg/kg and PRS-211,220 was tested at 5 mg/kg. Each treatment group was composed of at least fourteen animals.

Seven days following each MPTP injection, the animals were submitted to a functional test using the rotarod apparatus as described by Rozas et al. (Rozas G. et al., J. of Neuroscience Methods 83: 165-75, 1998). The performance of the animals in the rotarod system, which measures overall locomotor ability, reflects their ability to achieve functions generally affected in PD. The animals were trained for 4 days before beginning the experiment. Their task was to stay on the accelerating rod without falling for 12 minutes (3 minutes at each speed). The tested speeds were: 15, 19, 23 and 27 rpm. Animal performance on the rod was scored as follows: each animal could obtain a maximum of 3 points (1 for each minute) for full walking on the rod at each speed. Therefore, an animal could get a maximum score of 12 points (3 for each speed). Catching the circling beam of the rod without walking subtracted 0.5 points for every 3 circles circled by the animal. The first 3 circles did not affect the score. Fifty-two days following initiation of study animals were euthanized by i.p. administration of 100 mg/kg pentobarbitone sodium (CTS, Israel).

The results are shown in FIG. 8B, where we can see that each additional MPTP injection generate a further decrease in the animals ability to perform the rotarod test. Though this trend can be observed as early as in the first session following the first MPTP injection, the effect of MPTP on animal performance becomes statistically significant only from the third session and on. Indeed in the two first sessions we did not observe a significant effect of any of the treatment group on the rotarod score of the animals (data not shown). In the following sessions, we observe that the vehicle has no effect of its own, that dexanabinol at 20 mg/kg shows a positive trend at the fourth session and that PRS-211,220 at 5 mg/kg yields a statistically significant improvement in functional outcome at both later sessions when compared to vehicle.

Another parameter that was measured in this experiment is mortality, which was relatively high in this model. Animals that received only saline, and no MPTP, displayed, as expected, 0% mortality (0/14 animals). However, MPTP injected animals displayed 69% mortality (11/16 animals) over the period of the study, reflecting the severity of the model. Treatment with 20 mg/kg dexanabinol dramatically lowered this figure down to 11% mortality (1/9 animals), while treatment with 5 mg/kg PRS-211,220 was even more effective with 0% mortality (0/7 animals).

Altogether the results of this study show that the active compounds of this invention, which act through modulation of pro/anti-inflammatory mediators, may be therapeutically effective against the neuroinflammatory component of neurodegenerative diseases. The effect of these compounds is not only major on the mortality rate but above a certain level of TH-IR cells rescue, also significant on the functional abilities of the survivors.

### Example 16

### Peripheral Noxious Pain: the Formalin Test

Pain mediated by the peripheral nervous system, is tested in the ‘formalin test’ for cutaneous (peripheral) pain (Tjolson A. et al, Pain 51: 5-17, 1992). First the test compounds are injected i.p. Then formalin is injected s.c. in the plantar surface of the hind paw of a mouse 90 min after the test compound. Immediately after formalin administration pain is assessed (every 5 min for 1 hr) by the number of times the animal licks the formalin-injected paw.

### Example 17

### Neuropathic Pain: Attenuation of Mechanical Allodynia

The aim of this study is to assess the potential analgesic effects of our compounds in an animal model of neuropathic pain. A peripheral monopathy was induced in the right hind limb of rats following a chronic constriction of the sciatic nerve (Bennet G. J. and Xie Y-K. Pain 33: 87-107, 1988). The development of mechanical allodyna was monitored using an established behavioral test (Von Frey filaments).

Pre-surgery baseline values are ascertained as the mean of two pre-surgery values. Once the baseline values had been established, the animals are surgically prepared by constricting the right sciatic nerve with 4 chromic cat gut loose ligatures. On day 11 post-operation, the animals that have developed mechanical allodyna are arbitrarily allocated to the various treatment groups based on the pre-surgery values.

The design is randomized, performed in a masked fashion as to whether drug or vehicle is being given. The animals, male Sprague-Dawley rats, are allowed to acclimatize to the behavioral testing equipment before testing. On the testing day, the animals are given a single dose of one of the test compounds in a volume of 2.5 ml/kg. Following 15 and 180 minutes a series of Von Frey filaments (pre-calibrated before testing) are applied to the plantar surface of the hind paw, from below. The filaments are applied in ascending order starting with the weakest force; 0.37 g or filament handle no. 3.61), and the withdrawal threshold for both the ipsilateral and contralateral hind paws is evaluated. Each filament is indented on the mid-plantar surface of the foot to the point where it just starts to bend; this is repeated approximately 8-10 times per filament at a frequency of approximately 1 Hz. The withdrawal threshold is defined as being the lowest force of two or more consecutive Von Frey's filaments to elicit a reflex withdrawal response (i.e. a brief paw flick) and is measured in grams.

### Example 18

### Visceral Pain

The ability of the tricyclic dextrocannabinoid to decrease visceral pain is tested using the acetic acid induced model. ICR male mice (average body weight 30 g, Harlan, Israel) are pre-treated with i.v. injection of either vehicle (Cremophor:Ethanol 70:30 w/w further diluted 1:20 into saline; 5 ml/kg), or test compounds. Each treatment group comprises at least five animals. Fifteen minutes later, the mice are injected i.p. with 10 ml/kg of 0.6% acetic acid in water and the number of writhes are counted during a 5 minutes period, starting 5 minutes after the acetic acid administration. A writhing is considered as contraction of the abdominal muscles accompanied by an elongation of the body and extension of the hind limb. Results are expressed as mean number of writhes ±SEM. Data was analyzed using analysis of variance (ANOVA) followed by Tukey's post hoc test. A value of p<0.05 was considered statistically significant.

### Example 19

Diabetes Type I: the NOD Mice Model

The purpose of the present study is to establish a model in non-obese diabetic (NOD) mice to test the protective activity of dexanabinol and its analogs in an experimental setup relevant to human insulin-dependent diabetes mellitus.

NOD/It female mice (70-80 days old at study onset, Harlan, Israel) are weighted at day 1. Their baseline glucose level is established using a drop of blood obtained by sectioning the tip of the tail and a glucometer with the appropriate glucosticks (Elite, Bayer). Mice are then injected i.p. with cyclophosphamide (Sigma) diluted in saline at a dose of 300 mg/kg. The appearance of glucose in the urine of the animals is monitored every two days using a urine multistick (Bayer). When this test indicates that the animals reach glucourea, then the level of glucose in the blood is reassessed during two consecutive days after overnight starvation. Animals are defined as diabetic if their glucose blood levels are above 300 mg/dl. Three days following the diagnostic of diabetes, the animals are sacrificed by i.p. injection of 100 mg/kg pentobarbitone. Their spleen and pancreas are removed for further study including FACS analysis of the T cells subpopulations in the spleen and histo- and immuno-pathological evaluation of the pancreas.

The histopathological evaluation is carried out on ten Langerhans islands for each animal and the scoring is according to the following method (Sempe P. et al, Eur. J. Immunol. 21: 1163-9, 1991). The severity of the damage is scored according to the level of mononuclear infiltrate: 0—no infiltration, 1—periductular infiltrate, 2—peri-islet infiltrate, 3—intra-islet infiltrate, 4—intra-islet infiltrate associated with β-cell destruction. The mean score for the pancreas of each animal is calculated by dividing the total score by the number of islets examined.

### Example 20

### Renal Ischemia

The purpose of the present study is to test the nephro-protective activity of dexanabinol and its analogs in an acute renal ischemia model in rats.

Male Sprague Dawley rats (250 gr average body weight, Harlan, Israel) are anesthetized with a combination of xylazine and pentobarbitone 8 and 35 mg/kg i.p. respectively. Then a 45-minutes ischemia is induced bilaterally on both kidneys. The sedated animals are positioned on their backs. The abdomen skin is shaved and cleaned with 70% ethanol. A midline skin incision is performed (2-3 cm long) and the abdomen is opened through an incision in the linea Alba. The kidneys are explored after gentle removal of the intestines to the opposite direction. While this is done, the intestines are covered with wet (warm saline 37° C.) sterile sponges. The renal arteries are isolated by blunt dissection from the surrounding fat, and occluded together with the renal veins in the kidney hilus by arterial micro clips (FST Canada). Kidneys that become pale immediately after artery occlusion are considered ischemic. Only animals showing that both kidneys are ischemic are included in the study. During the ischemic insult the intestines are returned into the abdominal cavity. The wound is covered with wet sponges (they were kept wet by rinsing warm saline).In addition, rectal temperature is monitored to remain between 37° C.-38° C. Rectal temperature is measured using a thermistor (YSI USA model 400) and a measuring unit (Cole Parmer model 8402-00).

Forty-five minutes after the ischemia initiation, the artery clips are removed. Reperfusion is verified by the return of the pink color of the kidney. The wound is then closed with 3-0 silk suture material (Assut, Switzerland) in two layers (abdomen wall and skin). At 1, 3 and 7 days post ischemic insult animals are lightly anesthetized in an anesthesia chamber with ether and blood samples are collected after an infra orbital sinus puncture. Blood is collected into eppendorf tubes, and centrifuged (4000 rpm for 5 minutes). Serum is then separated and kept at −20° C. before evaluation of blood levels of creatinine and blood urea nitrogen (BUN). At the end of the study, animals are euthanized with pentobarbitone sodium 100 mg/kg i.p. Kidneys are removed, weighted and kept in 4% formaldehyde solution for possible further usage.

Treatments are administered i.v. into the femoral vein at 5 ml/kg to 10 animals per group, immediately after the end of the ischemic insult. Results are compared to ischemic (vehicle treated) and sham (the same procedure, without renal artery occlusion).

Statistical Analysis

The blood levels of BUN and creatinine are compared using ANOVA followed by Duncan's post-hoc test.

### Example 21

### Inflammatory Bowel Disease: The Acetic Acid-induced Model

The purpose of this study is to evaluate the activity of test compounds in a masked study of acetic acid-induced inflammatory bowel disease in rats.

Male Sprague Dawley rats (10 weeks old, 200-250 gr, Harlan, Israel) are lightly anaesthetized by i.p. injection of a ketamine:rompun combination (100:10 mg/kg respectively). A polyethylene catheter (outer diameter 1.7 mm) is inserted through the rectum 5 cm into the colon. And 2 ml of 5% acetic acid are then slowly administered into the colon. Fifteen seconds later the colon is washed with 3 ml saline and 15 seconds later with additional 3 ml of saline. Immediately after, each group of animals are treated with either one of the appropriate treatments. All treatments are administered once daily for 7 days. Animals are clinically followed for 1 week. During this period, the following parameters are daily monitored and recorded: body weight, presence of blood in the stool and stool consistency. These findings are scored according to table 1 (Murthy S. N. et al Dig. Dis. Sci. 38: 1722-34, 1993).

TABLE 1
Criteria for Scoring Disease Activity Index (DAI^{#}) of IBD.

| | Weight Loss | | Occult Blood or |
|---|---|---|---|
| Score | (%) | Stool Consistency* | Gross Bleeding |
| 0 | None | Normal | Negative |
| 1 | 1-5 | Loose Stool | Negative |
| 2 | 5-10 | Loose Stool | Hemoccult Positive |
| 3 | 10-15 | Diarrhea | Hemoccult Positive |
| 4 | >15 | Diarrhea | Gross Bleeding |

^{#}DAI - (combined score of weight loss, stool consistency, and bleeding)/3.
*Normal stool - well formed pellets; loose stools - pasty stool that does not stick to the anus; and diarrhea - liquid stools that sticks to the anus.

Seven days post disease induction animals are sacrificed with pentobarbital 100 mg/kg i.p. The whole colon is excised, slit longitudinally and examined under a magnifying glass, and any visible damage is recorded and scored according to table 2 (Wong et al, J. Pharm. Exp. Ther. 274: 475-80, 1995).

TABLE 2
Gross Pathology Scoring Method for Evaluating the Severity of IBD.

| Score | Pathology |
|---|---|
| 0 | No damage |
| 1 | Localized hyperemia and/or edema |
| 2 | Two or sites of hyperemia and/or edema |
| 3 | Localized erosion |
| 4 | Localized ulcer |
| 5 | More then 1 site of erosion/or ulcer, or 1 erosion site or ulcer |
| | extending >2 cm along the length of the colon |

Statistical Analysis

The clinical outcome is analyzed using analysis of variance (ANOVA) followed by Duncan's post-hoc test. A non-parametric test (Wilcoxon Rank Sum Test) is used for evaluating the gross pathology findings.

### Example 22

### Experimental Autoimmune Diseases: CIA, EAE and DTH

Autoimmune diseases are associated with elevated levels of inflammatory cytokines. The rodent models most commonly studied are experimental allergic encephalomyelitis (EAE), a model for multiple sclerosis in the human, experimental autoimmune arthritis, a model for rheumatoid arthritis in the human and delayed type hypersensitivity (DTH), a model for allergic reactions in the human. EAE is an autoimmune neurological disease elicited by sensitization of the animals to myelin basic protein from the central nervous system, which is also known as basic encephalitogenic protein. Experimental autoimmune arthritis is induced in animals by immunization with collagen in complete Freund's adjuvant: the model is therefore named collagen induced arthritis (CIA). Delayed type hypersensitivity is induced by the application of dinitrofluorobenzene according to a strict time-schedule, therefore the model generated correspond to allergic contact dermatitis in the human. The purpose of the present study is to test the ability of our compounds to prevent or attenuate the clinical signs of these three autoimmune disease models.

Collagen Induced Arthritis

Adult DBA/1 male mice (20 g average body weight, Harlan, Israel), at least eight per treatment group are used in this study. Bovine collagen type 2 is dissolved in 0.05 M acetic acid at a concentration of 2 mg/ml by stirring ON at 4° C. The collagen solution is further emulsified in an equal volume of Complete Freund's Adjuvant (CFA). Each animal is administered with 100 μg collagen type 2 in 0.1 ml CFA emulsion. The collagen is administered s.c. at the base of the tail. Twenty-one day after priming, the mice receive an intradermal booster injection of 100 μg collagen in Incomplete Freund's adjuvant.

The volume of each hind paw is measured using a plethysmometer (Hugo Basill, Italy), and the thickness using a dial, constant pressure gauge, (Mitutoyo, Japan). Measurements are performed before collagen administration and every second day throughout the designated follow-up period. All treatments are administered intraperitoneally. At the end of the treatment period the animals are sacrificed with pentobarbital 100 mg/kg i.p.

Statistical Analysis

The differences between the severity of the paw swelling among various treatment groups are compared using analysis of variance ANOVA followed by post-hoc t-Test. A value of p<0.05 is considered to be statistically significant.

Experimental Autoimmune Encephalomyelitis

Various animal models of autoimmune encephalomyelitis are known in the art, depending on the method of induction, the strain of the animal and the antigen employed to induce the disease. The impact of the test compounds is tested in EAE using Lewis rats in which the onset of disease is observed by the appearance of clinical symptoms about 10 days after induction. The disease progresses and the clinical score increases and peaks around day 15 and spontaneous recovery is observed around day 18 after induction of the disease. The animals (at least 10 per test group at initiation of study) are maintained on a 12 hours light/12 hours dark regimen, at a constant temperature of 22° C., with food and water ad libitum. EAE is induced in these animals by immunization with purified guinea pig myelin basic protein emulsified in Complete Freund's Adjuvant. Guinea pig myelin basic protein (MBP) is prepared from spinal cord homogenates defatted with chloroform/ethanol and the isolated protein is purified using ion exchange chromatography. Each animal receives 50 μg of the purified protein. A solution of MBP (0.5 mg/ml) is emulsified with an equal volume of Complete Freund's Adjuvant containing 4 mg/ml of mycobacterium tuberculosis, and each animal receives 100 μl (50 μl in each hind foot pad).

Animals are treated with test compounds or vehicle control, administered intravenously in a volume of 5 ml/kg, for three consecutive days starting from the onset of the disease (˜ at day 10 following disease induction). Methyl prednisolone is used as positive control and it is administered daily for 5 consecutive days i.v. at 20 mg/kg starting from day of disease induction by MBP injection. The results are recorded as clinical score; score of 0 indicates a normal animal with no clinical signs, 1 indicates tail paralysis, 2 indicates paraplegia, 3 indicates quadriplegia, 4 indicates complete body paralysis and 5 indicates death.

Statistical Analysis

The differences between the severity of the clinical outcomes among various treatment groups was analyzed by analysis of variance (ANOVA) followed by post-hoc Fisher test. A value of p<0.05 is considered to be statistically significant.

Delayed Type Hypersensitivity in Mice Model

Adult female BALB/c mice (20 g average body weight, Harlan, Israel) were sensitized on day 0 and day 1 by application of 30 μl of 0.15% Dinitrofluorobenzene (DNFB) diluted in acetone on the shaved skin of the abdomen. On day 6 the animals were challenged by application of 10 μl of DNFB in acetone on one ear. The contralateral ear was not challenged but received the application of 10 μl acetone. Test compounds were administered at increasing doses from 0 to 15 mg/kg i.p. twice, the first injection was immediately after DNFB challenge (on day 6) and the second injection was 16 hours after challenge (on day 7). Each treatment group comprised at least 7 animals. Dexamethasone (DXM) was used as positive control. Ear thickness was determined (in 0.01 mm units) 24 hours after challenge (and 6 hours after second treatment on day 7) using a dial thickness gauge (Mitutoyo, Japan).

Results are analyzed as ear thickness of DNFB treated over DNFB untreated contralateral ear. The impact of the test compound is further assessed by comparing its mean impact on the animals of the treatment group to the response generated by the appropriate vehicle only.

Although the present invention has been described with respect to various specific embodiments presented thereof for the sake of illustration only, such specifically disclosed embodiments should not be considered limiting. Many other such embodiments will occur to those skilled in the art based upon applicants' disclosure herein, and applicants propose to be bound only by the spirit and scope of their invention as defined in the appended claims.

1. A method for preventing, alleviating or treating a disease or disorder by regulating pro and anti-inflammatory mediators selected from COX-2, IL-1β, IL-2, iNOS, TNF-α, MCP-1, IL-10, IL-6, SOCS-1 and SOCS-3, by administering to an individual in need thereof of a therapeutically effective amount of a pharmaceutical composition comprising as an active ingredient a compound of the general formula (I): 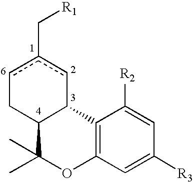
having the (3S,4S) configuration and being essentially free of the (3R,4R) enantiomer, wherein the dashed line indicates an optional C1-C2 or C6-C1 double bond, and wherein:
R₁ is selected from the group consisting of
a) R′ where R′ is selected from the group consisting of
A) a linear or branched, saturated or unsaturated, carbon side chain comprising 1-8 carbon atoms optionally interrupted by 1-3 heteroatoms, and
B) a saturated or unsaturated cyclic moiety, an aromatic moiety or a heterocyclic moiety; the cyclic moiety having from 5-20 atoms comprising one or two-ringed structures, wherein each ring comprises 3-8 carbons, optionally interrupted by 1-4 heteroatoms, and optionally further substituted with one or more groups selected from
i) a linear, branched or cyclic, saturated or unsaturated C₁-C₆ alkyl,
ii) a linear, branched or cyclic, saturated or unsaturated C₁-C₆ alkoxy,
iii) a linear, branched or cyclic, saturated or unsaturated C₁-C₆ alkylthio,
iv) a halogen,
v) carboxyl,
vi) —CO₂—C₁-C₄ alkyl, wherein the alkyl can be linear, branched or cyclic, saturated or unsaturated,
vii) keto,
viii) nitro,
ix) a saturated or unsaturated cyclic moiety, an aromatic or a heterocyclic moiety; the cyclic moiety having from 5-20 atoms comprising one or two-ringed structures, wherein each ring comprises 3-8 carbons, optionally interrupted by 1-4 heteroatoms, and optionally further substituted with one or more groups selected from i)-viii) as defined above,
b) an amine or an amide substituted with at least one substituent as defined in R′ above,
c) a thiol, a sulfide, a sulfoxide, a sulfone, a thioester or a thioamide optionally substituted with one substituent as defined in R′ above, and
d) a hydroxyl or an ether —OR′ wherein R′ is as defined above;
R₂ is selected from the group consisting of
a) a halogen,
b) a linear, branched or cyclic, saturated or unsaturated C₁-C₆-alkyl, and
c) —OR wherein R is selected from the group consisting of
A) -R″, wherein R″ is hydrogen or a linear, branched or cyclic, saturated or unsaturated C₁-C₆ alkyl optionally containing a terminal-OR′″ or OC(O)R′″ moiety wherein R′″ is hydrogen or a linear, branched or cyclic, saturated or unsaturated C₁-C₆ alkyl, and
B) —C(O)R′″ wherein R′″ is as previously defined; and
R₃ is selected from the group consisting of
a) a linear, branched or cyclic, saturated or unsaturated C₁-C₁₂ alkyl,
b) OR^{a}, in which R^{a} is a linear, branched or cyclic, saturated or unsaturated C₂-C₉ alkyl which may be substituted at the terminal carbon atom by a phenyl group, and
c) a linear, branched or cyclic, saturated or unsaturated C₁-C₇ alkyl-OR′″; wherein R′″ is as previously defined;
and pharmaceutically acceptable salts, esters or solvates thereof.

2. The method of claim 1, wherein the diseases characterized by abnormal production of any of COX-2, IL-1β, IL-2, iNOS, TNF-α, MCP-1, IL-10, IL-6, SOCS-1 or SOCS-3, are selected from the group consisting of inflammatory and immune disorders, pain, allergic inflammation, diseases characterized by monocyte infiltration such as sarcoidosis, Wegener's granulomatosis and tuberculosis, atherosclerosis, rheumatoid arthritis, vasculitis, interstitial lung disorders, inflammatory pulmonary diseases, asthma, inflammatory bowel diseases, pancreatitis, inflammatory skin diseases, osseous inflammation, tumor growth or metastasis, neurological diseases involving immune-mediated or post-traumatic inflammation, inflammatory demyelinating neuropathies, multiple sclerosis, neurodegenerative disorders such as Alzheimer's disease, Parkinson's disease, bacterial, parasitic or viral infections, sepsis, renal disorders, diabetic nephropathy, liver disorders, postoperative complications in cardiovasvular surgery, in transplants or organs or tissue replacements and in prosthetic implants, transplant rejection.

3. The method of claim 1, wherein the composition is administered orally, parenterally, intravenously, intramuscularly, intralesionally, subcutaneously, transdermally, intrathecally, rectally and intranasally.

4. The method of claim 1, wherein R₁ is OH, R₂ is OH, R₃ is 1,1-dimethylheptyl and there is a double bond between C6 and C1.

5. The method of claim 1, wherein R₁ is 2-mercaptoimidazole, R₂ is OH, R₃ is 1,1-dimethylheptyl and there is a double bond between C6 and C1.

6. The method of claim 1, wherein R₁ is imidazole, R₂ is OH, R₃ is 1,1-dimethylheptyl and there is a double bond between C6 and C1.

7. The method of claim 1, wherein R₁ is pyrazole, R₂ is OH, R₃ is 1,1-dimethylheptyl and there is a double bond between C6 and C1.

8. The method of claim 1, wherein R₁ is 4-methyl piperidine, R₂ is OH, R₃ is 1,1-dimethylheptyl and there is a double bond between C6 and C1.

9. The method of claim 1, wherein R₁ is 4-piperidino-piperidine, R₂ is OH, R₃ is 1,1-dimethylheptyl and there is a double bond between C6 and C1.

10. The method of claim 1, wherein the pharmaceutical composition further comprises a pharmaceutically acceptable diluent or carrier.

11. The method of claim 10, wherein the diluent comprises an aqueous cosolvent solution comprising a pharmaceutically acceptable cosolvent, a micellar solution or emulsion prepared with natural or synthetic ionic or non-ionic surfactants, or a combination of such cosolvent and micellar or emulsion solutions.

12. The method of claim 10, wherein the carrier comprises a solution of ethanol, a surfactant and water.

13. The method of claim 10, wherein the carrier composition is an emulsion comprising triglycerides, lecithin, glycerol, an emulsifier, and water.

14. The method of claim 1, wherein the pharmaceutical composition is in unit dosage form.

15. The method of claim 1, wherein the pharmaceutical composition is formulated for oral administration.

16. The method of claim 1, wherein the pharmaceutical composition is formulated for parenteral administration.

17. A method for decreasing transcription of at least one pro-inflammatory mediator selected from the group consisting of COX-2, IL-1β, IL-2, iNOS, TNF-α and MCP-1, which comprises administering to a subject in need of such treatment, a pharmaceutical composition comprising as an active ingredient a compound of the general formula (I): 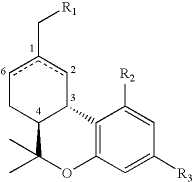
having the (3S,4S) configuration and being essentially free of the (3R,4R) enantiomer, wherein the dashed line indicates an optional C1-C2 or C6-C1 double bond, and wherein:
R₁ is selected from the group consisting of
e) R′ where R′ is selected from the group consisting of
A) a linear or branched, saturated or unsaturated, carbon side chain comprising 1-8 carbon atoms optionally interrupted by 1-3 heteroatoms, and
B) a saturated or unsaturated cyclic moiety, an aromatic moiety or a heterocyclic moiety; the cyclic moiety having from 5-20 atoms comprising one or two-ringed structures, wherein each ring comprises 3-8 carbons, optionally interrupted by 1-4 heteroatoms, and optionally further substituted with one or more groups selected from
i) a linear, branched or cyclic, saturated or unsaturated C₁-C₆ alkyl,
ii) a linear, branched or cyclic, saturated or unsaturated C₁-C₆ alkoxy,
iii) a linear, branched or cyclic, saturated or unsaturated C₁-C₆ alkylthio,
iv) a halogen,
v) carboxyl,
vi) —CO₂—C₁-C₄ alkyl, wherein the alkyl can be linear, branched or cyclic, saturated or unsaturated,
vii) keto,
viii) nitro,
ix) a saturated or unsaturated cyclic moiety, an aromatic or a heterocyclic moiety; the cyclic moiety having from 5-20 atoms comprising one or two-ringed structures, wherein each ring comprises 3-8 carbons, optionally interrupted by 1-4 heteroatoms, and optionally further substituted with one or more groups selected from i)-viii) as defined above,
f) an amine or an amide substituted with at least one substituent as defined in R′ above,
g) a thiol, a sulfide, a sulfoxide, a sulfone, a thioester or a thioamide optionally substituted with one substituent as defined in R′ above, and
h) a hydroxyl or an ether —OR′ wherein R′ is as defined above;
R₂ is selected from the group consisting of
d) a halogen,
e) a linear, branched or cyclic, saturated or unsaturated C₁-C₆-alkyl, and
f) —OR wherein R is selected from the group consisting of
A) -R″, wherein R″ is hydrogen or a linear, branched or cyclic, saturated or unsaturated C₁-C₆ alkyl optionally containing a terminal-OR′″ or OC(O)R′″ moiety wherein R′″ is hydrogen or a linear, branched or cyclic, saturated or unsaturated C₁-C₆ alkyl, and
B) —C(O)R′″ wherein R′″ is as previously defined; and
R₃ is selected from the group consisting of
d) a linear, branched or cyclic, saturated or unsaturated C₁-C₁₂ alkyl,
e) OR^{a}, in which R^{a} is a linear, branched or cyclic, saturated or unsaturated C₂-C₉ alkyl which may be substituted at the terminal carbon atom by a phenyl group, and
f) a linear, branched or cyclic, saturated or unsaturated C₁-C₇ alkyl-OR′″; wherein R′″ is as previously defined;
and pharmaceutically acceptable salts, esters or solvates thereof.

18. A method for increasing transcription of at least one of anti-inflammatory cytokine IL-10, protective cytokine IL-6 and a suppressor of cytokine signaling SOCS-1 or SOCS-3, which comprises administering to a subject in need thereof a pharmaceutical composition comprising as an active ingredient a compound of the general formula (I): 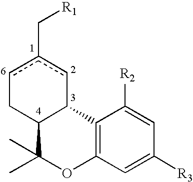
having the (3S,4S) configuration and being essentially free of the (3R,4R) enantiomer, wherein the dashed line indicates an optional C1-C2 or C6-C1 double bond, and wherein:
R₁ is selected from the group consisting of
i) R′ where R′ is selected from the group consisting of
A) a linear or branched, saturated or unsaturated, carbon side chain comprising 1-8 carbon atoms optionally interrupted by 1-3 heteroatoms, and
B) a saturated or unsaturated cyclic moiety, an aromatic moiety or a heterocyclic moiety; the cyclic moiety having from 5-20 atoms comprising one or two-ringed structures, wherein each ring comprises 3-8 carbons, optionally interrupted by 1-4 heteroatoms, and optionally further substituted with one or more groups selected from
i) a linear, branched or cyclic, saturated or unsaturated C₁-C₆ alkyl,
ii) a linear, branched or cyclic, saturated or unsaturated C₁-C₆ alkoxy,
iii) a linear, branched or cyclic, saturated or unsaturated C₁-C₆ alkylthio,
iv) a halogen,
v) carboxyl,
vi) —CO₂—C₁-C₄ alkyl, wherein the alkyl can be linear, branched or cyclic, saturated or unsaturated,
vii) keto,
viii) nitro,
ix) a saturated or unsaturated cyclic moiety, an aromatic or a heterocyclic moiety; the cyclic moiety having from 5-20 atoms comprising one or two-ringed structures, wherein each ring comprises 3-8 carbons, optionally interrupted by 1-4 heteroatoms, and optionally further substituted with one or more groups selected from i)-viii) as defined above,
j) an amine or an amide substituted with at least one substituent as defined in R′ above,
k) a thiol, a sulfide, a sulfoxide, a sulfone, a thioester or a thioamide optionally substituted with one substituent as defined in R′ above, and
l) a hydroxyl or an ether —OR′ wherein R′ is as defined above;
R₂ is selected from the group consisting of
g) a halogen,
h) a linear, branched or cyclic, saturated or unsaturated C₁-C₆-alkyl, and
i) —OR wherein R is selected from the group consisting of
A) -R″, wherein R″ is hydrogen or a linear, branched or cyclic, saturated or unsaturated C₁-C₆ alkyl optionally containing a terminal-OR′″ or OC(O)R′″ moiety wherein R′″ is hydrogen or a linear, branched or cyclic, saturated or unsaturated C₁-C₆ alkyl, and
B) —C(O)R′″ wherein R′″ is as previously defined; and
R₃ is selected from the group consisting of
g) a linear, branched or cyclic, saturated or unsaturated C₁-C₁₂ alkyl,
h) OR^{a}, in which R^{a} is a linear, branched or cyclic, saturated or unsaturated C₂-C₉ alkyl which may be substituted at the terminal carbon atom by a phenyl group, and
i) a linear, branched or cyclic, saturated or unsaturated C₁-C₇ alkyl-OR′″; wherein R′″ is as previously defined;
and pharmaceutically acceptable salts, esters or solvates thereof.


SEQUENCE LISTING (36 sequences)

SEQ ID NO: 1 - DNA, 21 bp, Artificial sequence
  Single strand DNA oligonucleotide
ttccgtttctcgtggtcactt

SEQ ID NO: 2 - DNA, 20 bp, Artificial sequence
  Single strand DNA oligonucleotide
agcgctgaggttttcctgaa

SEQ ID NO: 3 - DNA, 20 bp, Artificial sequence
  Single strand DNA oligonucleotide
gctgggatcctgagctttga

SEQ ID NO: 4 - DNA, 20 bp, Artificial sequence
  Single strand DNA oligonucleotide
ttggccatggtttcctgaac

SEQ ID NO: 5 - DNA, 21 bp, Artificial sequence
  Single strand DNA oligonucleotide
acactccttagtcctcggcca

SEQ ID NO: 6 - DNA, 20 bp, Artificial sequence
  Single strand DNA oligonucleotide
ccatcagaggcaaggaggaa

SEQ ID NO: 7 - DNA, 21 bp, Artificial sequence
  Single strand DNA oligonucleotide
gaaactccccaggatgctcac

SEQ ID NO: 8 - DNA, 19 bp, Artificial sequence
  Single strand DNA oligonucleotide
gccgcagaggtccaagttc

SEQ ID NO: 9 - DNA, 22 bp, Artificial sequence
  Single strand DNA oligonucleotide
agaaggagtggctaaggaccaa

SEQ ID NO: 10 - DNA, 21 bp, Artificial sequence
  Single strand DNA oligonucleotide
ggcataacgcactaggtttgc

SEQ ID NO: 11 - DNA, 21 bp, Artificial sequence
  Single strand DNA oligonucleotide
gccctttgctatggtgtcctt

SEQ ID NO: 12 - DNA, 20 bp, Artificial sequence
  Single strand DNA oligonucleotide
tccctggtttctcttcccaa

SEQ ID NO: 13 - DNA, 19 bp, Artificial sequence
  Single strand DNA oligonucleotide
ttcacctcactgtggccgt

SEQ ID NO: 14 - DNA, 20 bp, Artificial sequence
  Single strand DNA oligonucleotide
gcactctcttgcggaccatc

SEQ ID NO: 15 - DNA, 17 bp, Artificial sequence
  Single strand DNA oligonucleotide
tcacagttgccggctgg

SEQ ID NO: 16 - DNA, 20 bp, Artificial sequence
  Single strand DNA oligonucleotide
tctttgggacacctgctgct

SEQ ID NO: 17 - DNA, 21 bp, Artificial sequence
  Single strand DNA oligonucleotide
cagaagttcacgggacatgga

SEQ ID NO: 18 - DNA, 19 bp, Artificial sequence
  Single strand DNA oligonucleotide
ccagcaggtcggaagtggt

SEQ ID NO: 19 - DNA, 22 bp, Artificial sequence
  Single strand DNA oligonucleotide
gcatccctcttaacccggtact

SEQ ID NO: 20 - DNA, 19 bp, Artificial sequence
  Single strand DNA oligonucleotide
aataaggcgcccccactta

SEQ ID NO: 21 - DNA, 19 bp, Artificial sequence
  Single strand DNA oligonucleotide
aggcactccccgggagtac

SEQ ID NO: 22 - DNA, 19 bp, Artificial sequence
  Single strand DNA oligonucleotide
ggccacgttggaggagaga

SEQ ID NO: 23 - DNA, 21 bp, Artificial sequence
  Single strand DNA oligonucleotide
aaggactcaaatgggctttcc

SEQ ID NO: 24 - DNA, 23 bp, Artificial sequence
  Single strand DNA oligonucleotide
cctcattctgagacagaggcaac

SEQ ID NO: 25 - DNA, 20 bp, Artificial sequence
  Single strand DNA oligonucleotide
tcgccattgccaaggagtag

SEQ ID NO: 26 - DNA, 20 bp, Artificial sequence
  Single strand DNA oligonucleotide
ggtcaccccatcagatggaa

SEQ ID NO: 27 - DNA, 21 bp, Artificial sequence
  Single strand DNA oligonucleotide
ggttgtctcctgcgacttcaa

SEQ ID NO: 28 - DNA, 20 bp, Artificial sequence
  Single strand DNA oligonucleotide
gtaggccatgaggtccacca

SEQ ID NO: 29 - DNA, 20 bp, Artificial sequence
  Single strand DNA oligonucleotide
tcctgcctactggaagccaa

SEQ ID NO: 30 - DNA, 22 bp, Artificial sequence
  Single strand DNA oligonucleotide
agcccttcacgttattgcagat

SEQ ID NO: 31 - DNA, 25 bp, Artificial sequence
  Single strand DNA oligonucleotide
gggacttaatcagcaatatcaacgt

SEQ ID NO: 32 - DNA, 25 bp, Artificial sequence
  Single strand DNA oligonucleotide
ttctacaatggttgctgtctcatct

SEQ ID NO: 33 - DNA, 19 bp, Artificial sequence
  Single strand DNA oligonucleotide
gcatacgggtcctggcatc

SEQ ID NO: 34 - DNA, 21 bp, Artificial sequence
  Single strand DNA oligonucleotide
tgccatccaaccactcagtct

SEQ ID NO: 35 - DNA, 20 bp, Artificial sequence
  Single strand DNA oligonucleotide
acccactcctccacctttga

SEQ ID NO: 36 - DNA, 22 bp, Artificial sequence
  Single strand DNA oligonucleotide
ctgttgctgtagccaaattcgt